(19) United States
(12) Patent Application Publication
(10) Pub. No.: US 2024/0120038 A1
(43) Pub. Date: Apr. 11, 2024

(54) MEDICAL SUPPORT DEVICE, OPERATION METHOD OF MEDICAL SUPPORT DEVICE, AND OPERATION PROGRAM OF MEDICAL SUPPORT DEVICE

(71) Applicant: FUJIFILM Corporation, Tokyo (JP)

(72) Inventor: Caihua WANG, Kanagawa (JP)

(73) Assignee: FUJIFILM Corporation, Tokyo (JP)

(21) Appl. No.: 18/389,695

(22) Filed: Dec. 19, 2023

Related U.S. Application Data
(63) Continuation of application No. PCT/JP2022/025625, filed on Jun. 27, 2022.

(30) Foreign Application Priority Data
Jun. 28, 2021 (JP) 2021-106862
Sep. 17, 2021 (JP) 2021-152142

(51) Int. Cl. G16H 10/20; G16H 50/20

(52) U.S. Cl. CPC G16H 10/20; G16H 50/20

(57) ABSTRACT

A medical support device includes: a processor; and a memory connected to or built into the processor, and the processor acquires target input data which is input data related to a disease of a subject candidate for a clinical trial of a drug, and a clinical trial period, inputs the target input data and the clinical trial period to a machine learning model trained using supervised training data including accumulated input data related to a disease at two or more points in time and a time interval of the input data, and causes the machine learning model to output a prediction result regarding the disease of the subject candidate in the clinical trial period, and outputs selection reference information for determining whether or not to select the subject candidate as a subject for the clinical trial, according to the prediction result.

### CROSS-REFERENCE TO RELATED APPLICATIONS

This application is a continuation application of International Application No. PCT/JP2022/025625 filed on Jun. 27, 2022, the disclosure of which is incorporated herein by reference in its entirety. Further, this application claims priority from Japanese Patent Applications No. 2021-106862 filed on Jun. 28, 2021, and No. 2021-152142 filed on Sep. 17, 2021, the disclosures of which are incorporated herein by reference in their entirety.

### BACKGROUND

### 1. Technical Field

The technology of the present disclosure relates to a medical support device, an operation method of a medical support device, and an operation program of a medical support device.

### 2. Description of the Related Art

With the advent of a full-fledged aging society, efforts are being made to develop drugs (hereinafter abbreviated as anti-dementia drugs) for preventing the onset of diseases such as dementia represented by Alzheimer's dementia or delaying the progression of dementia. The efficacy of the anti-dementia drug is evaluated after a clinical trial for a certain period of time, for example, for one year and six months (18 months). Subjects for this clinical trial are preferably people with a relatively rapid progression of dementia in order to correctly evaluate the efficacy of anti-dementia drugs. This is because, in the case of a person with a slow progression of dementia, it is not clear whether the progression is being suppressed by the efficacy of the anti-dementia drug, or whether the progression is delayed due to reasons specific to that person.

In that case, before conducting a clinical trial of an anti-dementia drug, it is necessary to predict people with a relatively rapid progression of dementia and select them as subjects for the clinical trial. A method using a machine learning model is available as a method of predicting people with a relatively rapid progression of dementia. For example, “M. Nguyen, T. He and L. An et al.: Predicting Alzheimer's disease progression using deep recurrent neural networks, NeuroImage, November 2020” (hereinafter referred to as Document 1) discloses a technology for predicting the progression of dementia using a recurrent neural network (RNN) as a machine learning model. In Document 1, test data related to dementia at three or more points in time (for example, test data three months ago, two months ago, and one month ago) is given to the RNN as a set of supervised training data for learning.

### SUMMARY

The number of donors of test data related to dementia is less than 3,000 even in Alzheimer's disease Neuroimaging Initiative (ADNI), which is the most popular database. That is, in the method of Document 1, the amount of supervised training data is significantly insufficient. Therefore, in the method of Document 1, there is a concern that overlearning occurs, the accuracy of predicting the progression of dementia significantly decreases, and a person who is unsuitable as the subject for a clinical trial of the anti-dementia drug may be selected.

An embodiment according to the technology of the present disclosure provides a medical support device, an operation method of a medical support device, and an operation program of a medical support device capable of selecting a person suitable as a subject for a clinical trial of a drug with high accuracy.

According to an aspect of the present disclosure, there is provided a medical support device comprising: a processor; and a memory connected to or built into the processor, in which the processor is configured to: acquire target input data which is input data related to a disease of a subject candidate for a clinical trial of a drug, and a clinical trial period; input the target input data and the clinical trial period to a machine learning model trained using supervised training data including accumulated input data related to a disease at two or more points in time and a time interval of the input data, and cause the machine learning model to output a prediction result regarding the disease of the subject candidate in the clinical trial period; and output selection reference information for determining whether or not to select the subject candidate as a subject for the clinical trial, according to the prediction result.

It is preferable that the time interval is an interval set according to the clinical trial period.

It is preferable that the input data includes at least one of test data indicating a result of a test related to a disease or diagnostic data indicating a result of a diagnosis related to the disease.

It is preferable that the machine learning model outputs, as the prediction result, a score quantitatively representing a degree of progression of a disease.

It is preferable that the machine learning model further outputs, as the prediction result, a class qualitatively representing the degree of progression of the disease.

It is preferable that, in addition to the supervised training data, clinical trial suitable data that satisfies an employment condition determined in advance according to the drug is provided, a prediction result for setting from the machine learning model is output by inputting input data and a time interval of the clinical trial suitable data to the machine learning model, and the processor is configured to output the selection reference information according to a selection condition set based on at least a prediction result distribution for setting, which is a distribution of the number of data items of the prediction result for setting.

It is preferable that the selection condition is set based on an exclusion group prediction result distribution, which is a distribution of the number of data items of the prediction result for setting of a group of persons to be excluded from the subject for the clinical trial, the group being extracted based on correct answer data included in the clinical trial suitable data.

It is preferable that the selection condition is set based on a selection group prediction result distribution, which is a distribution of the number of data items of the prediction result for setting of a group of persons to be selected as the subject for the clinical trial, the group being extracted based on correct answer data included in the clinical trial suitable data.

It is preferable that a plurality of provisional selection conditions are set in the prediction result distribution for setting, the number of errors in the prediction result for setting with respect to correct answer data is counted for each of the plurality of provisional selection conditions, and the provisional selection condition having a minimum number of errors is set as the selection condition.

It is preferable that the selection condition is set based on a correct answer data distribution, which is a distribution of the number of correct answer data items included in the clinical trial suitable data, in addition to the prediction result distribution for setting.

It is preferable that the selection condition is set by applying a provisional selection condition set in the correct answer data distribution to the prediction result distribution for setting.

It is preferable that the selection condition is set at a boundary of a region defined as including a person with a rapid progression of a disease in the prediction result distribution for setting.

It is preferable that the disease is dementia.

According to another aspect of the present disclosure, there is provided an operation method of a medical support device, the method comprising: acquiring target input data which is input data related to a disease of a subject candidate for a clinical trial of a drug, and a clinical trial period; inputting the target input data and the clinical trial period to a machine learning model trained using supervised training data including accumulated input data related to a disease at two or more points in time and a time interval of the input data, and causing the machine learning model to output a prediction result regarding the disease of the subject candidate in the clinical trial period; and outputting selection reference information for determining whether or not to select the subject candidate as a subject for the clinical trial, according to the prediction result.

According to another aspect of the present disclosure, there is provided an operation program of a medical support device causing a computer to execute a process comprising: acquiring target input data which is input data related to a disease of a subject candidate for a clinical trial of a drug, and a clinical trial period; inputting the target input data and the clinical trial period to a machine learning model trained using supervised training data including accumulated input data related to a disease at two or more points in time and a time interval of the input data, and causing the machine learning model to output a prediction result regarding the disease of the subject candidate in the clinical trial period; and outputting selection reference information for determining whether or not to select the subject candidate as a subject for the clinical trial, according to the prediction result.

According to the technology of the present disclosure, it is possible to provide a medical support device, an operation method of a medical support device, and an operation program of a medical support device capable of selecting a person suitable as a subject for a clinical trial of a drug with high accuracy.

### BRIEF DESCRIPTION OF THE DRAWINGS

Exemplary embodiments according to the technique of the present disclosure will be described in detail based on the following figures, wherein:

FIG. 1 is a diagram showing a clinical trial subject selection support server and a user terminal;

FIG. 2 is a diagram showing target input data;

FIG. 3 is a diagram showing a clinical trial period;

FIG. 4 is a table showing selection reference information;

FIG. 5 is a block diagram showing a computer constituting the clinical trial subject selection support server;

FIG. 6 is a block diagram showing a processing unit of a CPU of the clinical trial subject selection support server;

FIG. 7 is a block diagram showing a detailed configuration of a dementia progression prediction model;

FIG. 8 is a diagram showing an outline of processing in a learning phase of the dementia progression prediction model;

FIG. 9 is a diagram for describing the formation of supervised training data of the dementia progression prediction model;

FIG. 10 is a diagram for describing another example of the formation of supervised training data of the dementia progression prediction model;

FIG. 11 is a diagram showing an outline of processing in an operation phase of the dementia progression prediction model;

FIG. 12 is a diagram showing a selection condition;

FIG. 13 is a diagram showing selection reference information in a case where a selection condition is satisfied;

FIG. 14 is a diagram showing selection reference information in a case where a selection condition is not satisfied;

FIG. 15 is a diagram showing a clinical trial subject selection support screen;

FIG. 16 is a diagram showing a clinical trial subject selection support screen on which a message indicating selection reference information is displayed;

FIG. 17 is a diagram showing a clinical trial subject selection support screen on which a message indicating selection reference information of two subject candidates is displayed;

FIG. 18 is a flowchart showing a processing procedure of the clinical trial subject selection support server;

FIG. 19 is a diagram showing another example of a selection condition;

FIG. 20 is a diagram showing another example of a score prediction result;

FIG. 21 is a diagram showing still another example of a score prediction result;

FIG. 22 is a diagram showing another example of a progression prediction result;

FIG. 23 is a diagram showing a state in which supervised training data and clinical trial suitable data are generated from all data;

FIG. 24 is a diagram showing a state in which target input data for setting and a clinical trial period for setting of clinical trial suitable data are input to a dementia progression prediction model trained using supervised training data and a score prediction result for setting is output from the dementia progression prediction model;

FIG. 25 is a graph showing a correct answer score distribution for setting and a score prediction result distribution for setting;

FIG. 26 is a diagram showing Method 1 of setting a selection condition based on the correct answer score distribution for setting and the score prediction result distribution for setting;

FIG. 27 is a diagram showing a case where a selection condition set by Method 1 is satisfied;

FIG. 28 is a diagram showing a case where a selection condition set by Method 1 is not satisfied;

FIG. 29 is a diagram showing a state in which a score prediction result distribution for exclusion group setting is generated;

FIG. 30 is a diagram showing Method 2 of setting a selection condition based on the score prediction result distribution for exclusion group setting;

FIG. 31 is a diagram showing a state in which a score prediction result distribution for selection group setting is generated;

FIG. 32 is a diagram showing Method 3 of setting a selection condition based on the score prediction result distribution for selection group setting;

FIG. 33 is a diagram showing Method 4 of setting a plurality of provisional selection conditions in a score prediction result distribution for setting, calculating a rate of error in score prediction results for setting with respect to correct answer scores for setting for each of the plurality of provisional selection conditions, and setting a provisional selection condition with a minimum error rate as a selection condition; and

FIG. 34 is a diagram showing Method 5 of setting a selection condition at a boundary of a region defined as including a person with a rapid progression of dementia in the score prediction result distribution for setting.

### DETAILED DESCRIPTION

### First Embodiment

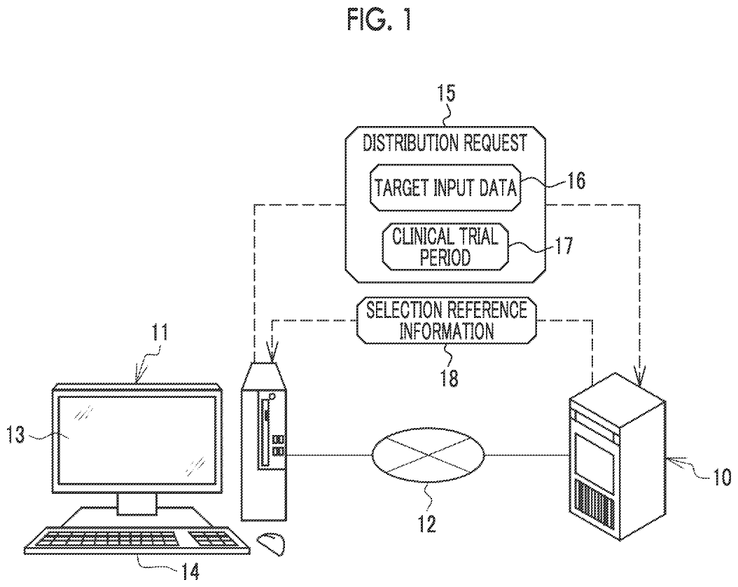

As shown in FIG. 1 as an example, a clinical trial subject selection support server 10 is connected to a user terminal 11 via a network 12. The clinical trial subject selection support server 10 is an example of a “medical support device” according to the technology of the present disclosure. The user terminal 11 is installed in, for example, a pharmaceutical development facility, and is operated by drug discovery staff involved in the development of drugs for preventing the onset of dementia or delaying the progression of dementia, particularly Alzheimer's dementia, that is, an anti-dementia drug, at the pharmaceutical development facility. Examples of dementia include Lewy body dementia, vascular dementia, and the like, in addition to Alzheimer's dementia. The anti-dementia drug may be used for Alzheimer's disease other than Alzheimer's dementia. Specifically, examples thereof include a preclinical Alzheimer's disease (PAD) and mild cognitive impairment (MCI) due to Alzheimer's disease. Hereinafter, Alzheimer's disease is sometimes abbreviated as AD. The disease is preferably a brain disease such as dementia as an example. The user terminal 11 includes a display 13 and an input device 14 such as a keyboard and a mouse. The network 12 is, for example, a wide area network (WAN) such as the Internet or a public communication network. Although only one user terminal 11 is connected to the clinical trial subject selection support server 10 in FIG. 1, in practice, a plurality of user terminals 11 of a plurality of pharmaceutical development facilities are connected to the clinical trial subject selection support server 10.

The user terminal 11 transmits a distribution request 15 to the clinical trial subject selection support server 10. The distribution request 15 includes target input data 16 and a clinical trial period 17. The distribution request 15 is a request for causing the clinical trial subject selection support server 10 to distribute selection reference information 18 referred to by the drug discovery staff in a case of selecting subjects for clinical trials of the anti-dementia drug under development.

The target input data 16 is input data related to dementia of a subject candidate, who is a candidate for a clinical trial subject, and is preferably data related to diagnostic criteria for dementia.

Diagnostic criteria for dementia include the diagnostic criteria described in “Dementia disease medical care guideline 2017” supervised by the Japanese Society of Neurology, “International Statistical Classification of Diseases and Related Health Problems (ICD)-11 (ICD-11)”, the American Psychiatric Association's “Diagnostic and Statistical Manual of Mental Disorders, Fifth Edition (DSM-5)”, and the “National Institute on Aging-Alzheimer's Association workgroup (NIA-AA) criteria”. Such diagnostic criteria can be cited, and the contents thereof are incorporated in the present specification.

Examples of data related to the diagnostic criteria for dementia include the data related to the above-described diagnostic criteria. The target input data 16 includes data related to diagnostic criteria for dementia. Specifically, data related to diagnostic criteria for dementia includes cognitive function test data, morphological image test data, brain function image test data, blood/cerebrospinal fluid test data, genetic test data, and the like. The target input data 16 preferably includes at least the morphological image test data, and more preferably includes at least the morphological image test data and the cognitive function test data.

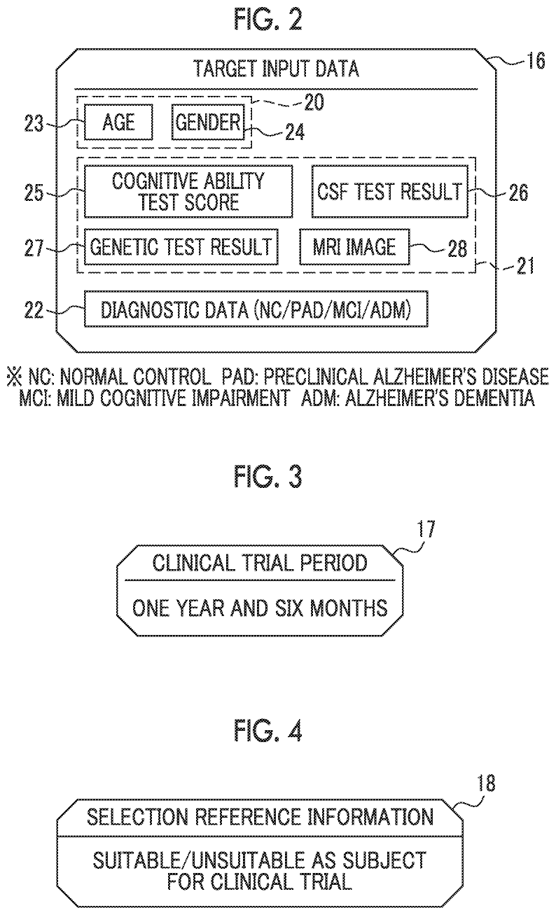

Cognitive function test data includes a clinical dementia rating-sum of boxes (hereinafter abbreviated as CDR-SOB) score, a mini-mental state examination (hereinafter abbreviated as MMSE) score, an Alzheimer's disease assessment scale-cognitive subscale (hereinafter abbreviated as ADAS-Cog) score, and the like. The morphological image test data includes a brain tomographic image obtained by magnetic resonance imaging (MRI) (hereinafter referred to as an MRI image) 28 (refer to FIG. 2), a tomographic image of the brain obtained by computed tomography (CT), and the like.

The brain function image test data include a tomographic image of the brain obtained by a positron emission tomography (PET) (hereinafter referred to as a PET image), a tomographic image of the brain obtained by a single photon emission computed tomography (SPECT), an image (hereinafter referred to as a SPECT image), and the like. The blood/cerebrospinal fluid test data includes an amount of phosphorylated tau protein (p-tau) 181 in cerebrospinal fluid (hereinafter abbreviated as CSF), and the like. The genetic test data includes a test result of a genotype of an ApoE gene, and the like.

The target input data 16 is input by the drug discovery staff operating the input device 14. Subject candidates are, for example, those recruited for clinical trials at pharmaceutical development facilities. The clinical trial period 17 is literally a period for conducting a clinical trial of the anti-dementia drug, and is set in advance according to the anti-dementia drug under development. Although not shown, the distribution request 15 also includes a terminal ID (identification data) or the like for uniquely identifying the user terminal 11 that is a transmission source of the distribution request 15.

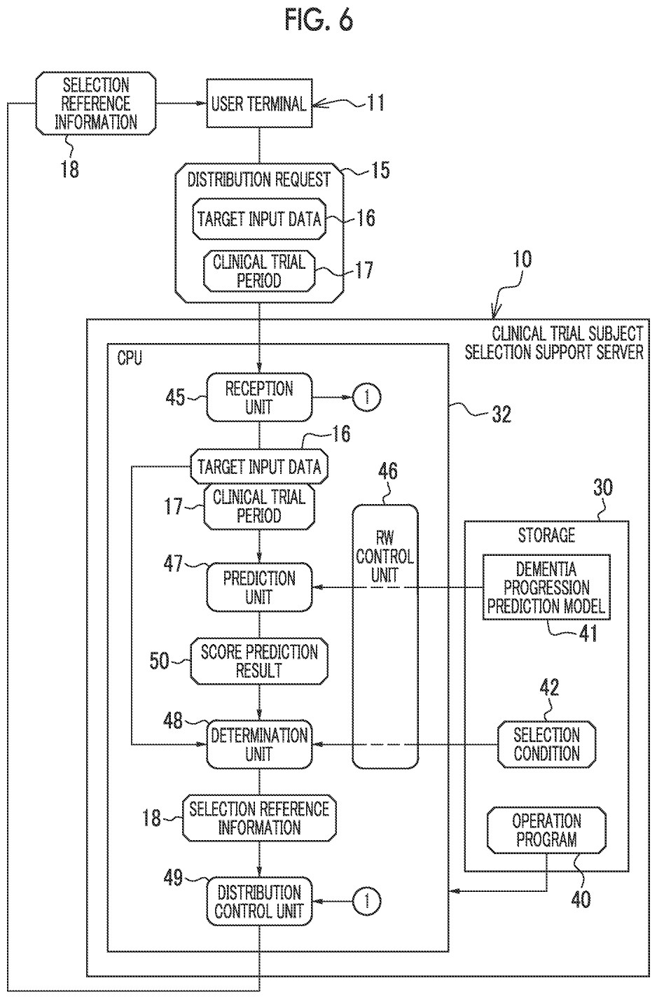

In a case where the distribution request 15 is received, the clinical trial subject selection support server 10 inputs the target input data 16 and the clinical trial period 17 to a dementia progression prediction model 41 (refer to FIG. 6), and causes the dementia progression prediction model 41 to output a prediction result regarding the dementia of the subject candidate. The clinical trial subject selection support server 10 generates the selection reference information 18 according to the prediction result and distributes the generated selection reference information 18 to the user terminal 11 that is the transmission source of the distribution request 15. In a case where the selection reference information 18 is received, the user terminal 11 displays the selection reference information 18 on the display 13 and provides the selection reference information 18 for viewing by drug discovery staff.

As shown in FIG. 2 as an example, the target input data 16 includes candidate data 20, test data 21, and diagnostic data 22. The candidate data 20 is data indicating attributes of a subject candidate, and includes an age 23 and a gender 24 of the subject candidate. The target input data 16 is, for example, data obtained on the same date as the transmission date of the distribution request 15. The target input data 16 may be data obtained on a transmission date of the distribution request 15 and a period from three days to one week before the transmission date. In addition, the target input data 16 may be data obtained on a clinical trial start date or a period from three days to one week before the clinical trial start date.

The test data 21 is data indicating a result of a test related to dementia of the subject candidate, and includes a cognitive ability test score 25 which is cognitive function test data, a cerebrospinal fluid (hereinafter abbreviated as CSF) test result 26 which is blood/cerebrospinal fluid test data, a genetic test result 27 which is a genetic test data, and the MM image 28 which is a morphological image test data. The cognitive ability test score 25 is, for example, a clinical dementia rating-sum of boxes (hereinafter abbreviated as CDR-SOB) score. The CSF test result 26 is, for example, the amount of phosphorylated tau protein (p-tau) 181 in CSF.

The genetic test result 27 is, for example, a test result of a genotype of the ApoE gene. The genotype of the ApoE gene is a combination of two types among three types of ApoE genes of ε2, ε3, and ε4 (ε2 and ε3, ε3 and ε4, and the like). A risk of developing Alzheimer's dementia in a person with a genotype including one or two of ε4 (ε2 and ε4, ε4 and ε4, and the like) is estimated to be about 3 to 12 times higher than that in a person with a genotype without ε4 (ε2 and ε3, ε3 and ε3, and the like).

The diagnostic data 22 is data indicating a result of diagnosis related to dementia of the subject candidate, which has been made by a doctor at the current point in time with reference to the test data 21 and the like. The diagnostic data 22 is any one of normal control (NC), preclinical AD (PAD), mild cognitive impairment (MCI), and Alzheimer's dementia (ADM). In this way, there are a plurality of types of target input data 16, and the dementia progression prediction model 41 is a so-called multimodal machine learning model.

As shown in FIG. 3 as an example, the clinical trial period 17 is one year and six months (18 months) in the present embodiment. The clinical trial period 17 varies depending on the anti-dementia drug, but is about one year to two years.

As shown in FIG. 4 as an example, the selection reference information 18 is either that the subject candidate is suitable/unsuitable as a subject for the clinical trial.

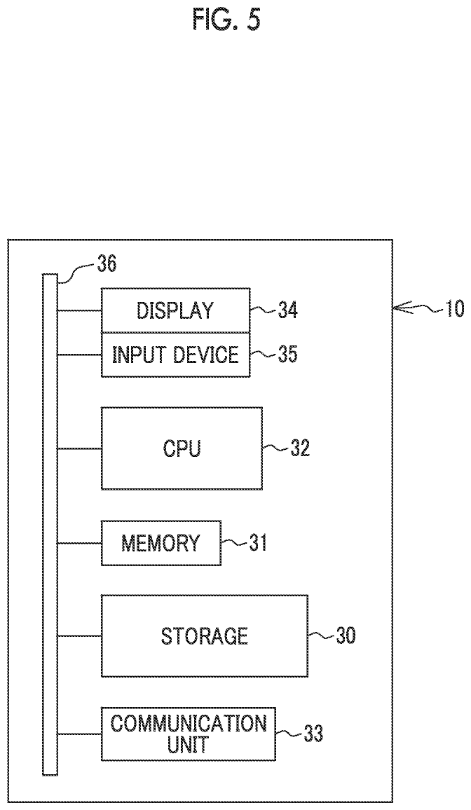

As shown in FIG. 5 as an example, a computer constituting the clinical trial subject selection support server 10 comprises a storage 30, a memory 31, a central processing unit (CPU) 32, a communication unit 33, a display 34, and an input device 35. These components are connected to each other through a bus line 36. Note that CPU 32 is an example of a “processor” according to the technology of the present disclosure.

The storage 30 is a hard disk drive built into the computer constituting the clinical trial subject selection support server 10 or connected via a cable or a network. Alternatively, the storage 30 is a disk array in which a plurality of hard disk drives are connected in series. The storage 30 stores a control program such as an operating system, various application programs, various types of data associated with these programs, and the like. A solid state drive may be used instead of the hard disk drive.

The memory 31 is a work memory for the CPU 32 to execute processing. The CPU 32 loads the program stored in the storage 30 into the memory 31 and executes processing corresponding to the program. Thus, the CPU 32 integrally controls the respective units of the computer. The memory 31 may be built into the CPU 32.

The communication unit 33 controls transmission of various types of information to and from an external device such as the user terminal 11. The display 34 displays various screens. Various screens have operation functions by a graphical user interface (GUI). The computer constituting the clinical trial subject selection support server 10 receives inputs of operation instructions from the input device 35 through various screens. The input device 35 is a keyboard, a mouse, a touch panel, a microphone for voice input, or the like.

As shown in FIG. 6 as an example, an operation program 40 is stored in the storage 30 of the clinical trial subject selection support server 10. The operation program 40 is an application program for causing the computer to function as the clinical trial subject selection support server 10. That is, the operation program 40 is an example of an “operation program of a medical support device” according to the technology of the present disclosure. The storage 30 also stores a dementia progression prediction model 41 and a selection condition 42. The dementia progression prediction model 41 is an example of a “machine learning model” according to the technology of the present disclosure.

In a case where the operation program 40 is activated, the CPU 32 of the computer constituting the clinical trial subject selection support server 10 cooperates with the memory 31 and the like to function as a reception unit 45, a read and write (hereinafter abbreviated as RW) control unit 46, a prediction unit 47, a determination unit 48, and a distribution control unit 49.

The reception unit 45 receives the distribution request 15 from the user terminal 11. Since the distribution request 15 includes the target input data 16 and the clinical trial period 17 as described above, the reception unit 45 receives the distribution request 15 to acquire the target input data 16 and the clinical trial period 17. The reception unit 45 outputs the target input data 16 and the clinical trial period 17 to the prediction unit 47. The reception unit 45 also outputs the cognitive ability test score 25 of the target input data 16 to the determination unit 48. Furthermore, the reception unit 45 outputs a terminal ID of the user terminal 11 (not shown) to the distribution control unit 49.

The RW control unit 46 controls storage of various types of data in the storage 30 and reading out of various types of data in the storage 30. For example, the RW control unit 46 reads out the dementia progression prediction model 41 from the storage 30 and outputs the dementia progression prediction model 41 to the prediction unit 47. Furthermore, the RW control unit 46 reads out the selection condition 42 from the storage 30 and outputs the selection condition 42 to the determination unit 48.

The prediction unit 47 inputs the target input data 16 and the clinical trial period 17 to the dementia progression prediction model 41, and causes the dementia progression prediction model 41 to output a score prediction result 50. The prediction unit 47 outputs the score prediction result 50 to the determination unit 48. The score prediction result 50 is an example of a “prediction result” and a “score quantitatively representing a degree of progression of dementia” according to the technology of the present disclosure.

The determination unit 48 determines whether or not the subject candidate is suitable as a subject for the clinical trial according to the cognitive ability test score 25 from the reception unit 45 and the score prediction result 50 from the prediction unit 47, in accordance with the selection condition 42. The determination unit 48 generates the selection reference information 18 based on the determination result and outputs the generated selection reference information 18 to the distribution control unit 49.

The distribution control unit 49 performs control to distribute the selection reference information 18 to the user terminal 11 that is a transmission source of the distribution request 15. In this case, the distribution control unit 49 specifies the user terminal 11 that is the transmission source of the distribution request 15 based on the terminal ID from the reception unit 45.

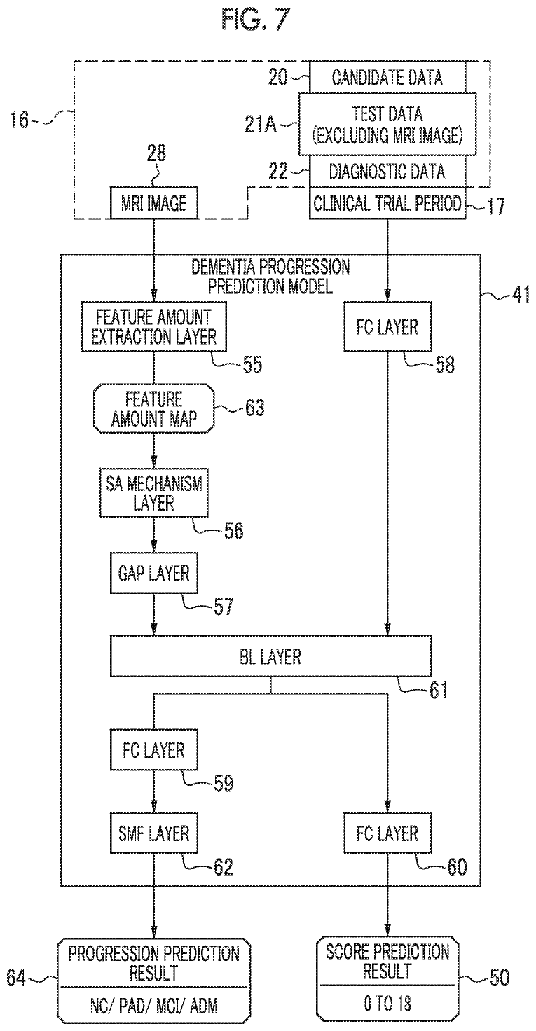

As shown in FIG. 7 as an example, the dementia progression prediction model 41 includes a feature amount extraction layer 55, a self-attention (hereinafter abbreviated as SA) mechanism layer 56, a global average pooling (hereinafter abbreviated as GAP) layer 57, fully connected (hereinafter abbreviated as FC) layers 58, 59, and 60, a bi-linear (hereinafter abbreviated as BL) layer 61, and a softmax function (hereinafter abbreviated as SMF) layer 62.

The feature amount extraction layer 55 is, for example, Densely Connected Convolutional Networks (DenseNet). The MRI image 28 is input to the feature amount extraction layer 55. The feature amount extraction layer 55 performs convolution processing or the like on the MRI image 28 to convert the MRI image 28 into a feature amount map 63. The feature amount extraction layer 55 outputs the feature amount map 63 to the SA mechanism layer 56.

The SA mechanism layer 56 performs convolution processing on the feature amount map 63 while changing the coefficients of a convolution filter according to the feature amount of the feature amount map 63 to be processed. The convolution processing performed by the SA mechanism layer 56 is hereinafter referred to as SA convolution processing. The SA mechanism layer 56 outputs the feature amount map 63 after the SA convolution processing to the GAP layer 57.

The GAP layer 57 performs global average pooling processing on the feature amount map 63 after the SA convolution processing. The global average pooling processing is processing of obtaining an average value of feature amounts for each channel of the feature amount map 63. For example, in a case where the number of channels of the feature amount map 63 is 512, an average value of 512 feature amounts is obtained by the global average pooling processing. The GAP layer 57 outputs the obtained average value of the feature amounts to the BL layer 61.

The candidate data 20, test data 21A excluding the MRI image 28, the diagnostic data 22, and the clinical trial period 17 are input to the FC layer 58. The gender 24 of the candidate data 20 is input as a numerical value such as 1 for male and 0 for female. Similarly, the genetic test result 27 of the test data 21 is input as a numerical value such as 1 for the combination of ε2 and ε3 and 2 for the combination of ε3 and ε3. The diagnostic data 22 is similarly input as a numerical value. The FC layer 58 has an input layer including units corresponding to the number of data items and an output layer including units corresponding to the number of data items handled by the BL layer 61. Each unit of the input layer and each unit of the output layer are fully connected to each other, and weights are set for each unit. The candidate data 20, the test data 21A excluding the MRI image 28, the diagnostic data 22, and the clinical trial period 17 are input to each unit of the input layer. The product sum of the each piece of the data and the weight which is set for each unit is an output value of each unit of the output layer. The FC layer 58 outputs the output value of the output layer to the BL layer 61.

The BL layer 61 performs bi-linear processing on the average value of the feature amounts from the GAP layer 57 and the output value from the FC layer 58. The BL layer 61 outputs the values after the bi-linear processing to the FC layers 59 and 60. For the BL layer 61 and the bi-linear processing, the following document can be referred to.
<Goto, T. etc, multi-modal deep learning for predicting progression of Alzheimer's disease using bi-linear shake fusion, Proc. SPIE 11314, Medical Imaging (2020)>

The FC layer 59 converts the values after the bi-linear processing into variables handled by the SMF of the SMF layer 62. Similarly to the FC layer 58, the FC layer 59 has an input layer including units corresponding to the number of values after the bi-linear processing and an output layer including units corresponding to the number of variables handled by the SMF. Each unit of the input layer and each unit of the output layer are fully connected to each other, and weights are set for each unit. A value after the bi-linear processing is input to each unit of the input layer. The product sum of the value after the bi-linear processing and the weight which is set for each unit is an output value of each unit of the output layer. This output value is a variable handled by the SMF. The FC layer 59 outputs variables handled by the SMF to the SMF layer 62. The SMF layer 62 outputs a progression prediction result 64 by applying the variables to the SMF. As in the diagnostic data 22, the progression prediction result 64 is a content indicating whether the subject candidate is normal control, preclinical AD, mild cognitive impairment, or Alzheimer's dementia. The progression prediction result 64 is an example of a “prediction result” and a “class qualitatively representing a degree of progression of dementia” according to the technology of the present disclosure.

The FC layer 60 converts the values after the bi-linear processing into the score prediction result 50. Similarly to the FC layers 58 and 59, the FC layer 60 has an input layer including units corresponding to the number of values after the bi-linear processing, and an output layer of the score prediction result 50. Each unit of the input layer and the output layer are fully connected to each other, and weights are set for each. A value after the bi-linear processing is input to each unit of the input layer. The product sum of the value after the bi-linear processing and the weight which is set for each unit is an output value of the output layer. This output value is the score prediction result 50. The score prediction result 50 is a prediction result of the score itself of the cognitive ability test of the subject candidate, here the CDR-SOB score itself, at the end point in time of the clinical trial period 17. The CDR-SOB score takes a value of 0 to 18, where 0 is normal control and 18 is the maximum cognitive impairment. In this way, the dementia progression prediction model 41 is a so-called multi-task machine learning model that outputs the progression prediction result 64 and the score prediction result 50.

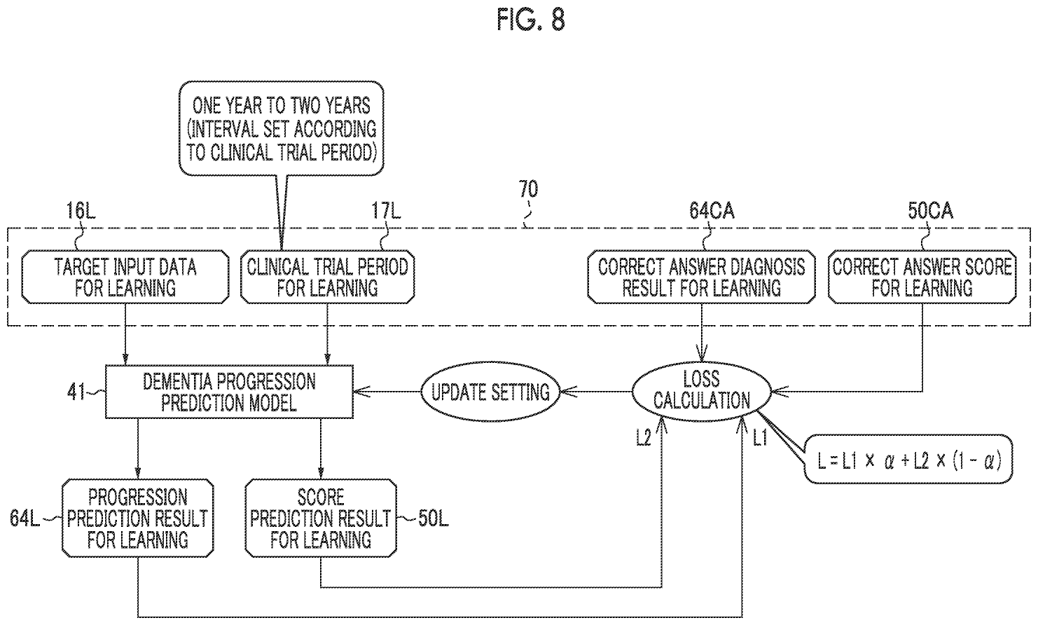

As shown in FIG. 8 as an example, the dementia progression prediction model 41 is trained by being giving supervised training data (also referred to as training data or learning data) 70 in a learning phase. The supervised training data 70 is a set of target input data for learning 16L, a clinical trial period for learning 17L, a correct answer diagnosis result for learning 64CA, and a correct answer score for learning 50CA. The target input data for learning 16L is, for example, the target input data 16 of a certain sample subject (including a patient, the same applies hereinafter) accumulated in a database such as ADNI at the start point in time of the clinical trial period for learning 17L. The clinical trial period for learning 17L is an interval set according to the clinical trial period 17. The clinical trial period for learning 17L is one year to two years in this example. The period of one year to two years is a period of six months plus or minus one year and six months of the clinical trial period 17.

The correct answer diagnosis result for learning 64CA is a diagnosis result of dementia that is actually given to the sample subject by the doctor at the end point in time of the clinical trial period for learning 17L. The correct answer score for learning 50CA is a score of a cognitive ability test that is actually performed by the sample subject at the end point in time of the clinical trial period for learning 17L. The target input data for learning 16L is an example of “accumulated input data related to dementia at two or more points in time” according to the technology of the present disclosure. Further, the clinical trial period for learning 17L is an example of a “time interval of the input data” according to the technology of the present disclosure.

In the learning phase, the target input data for learning 16L and the clinical trial period for learning 17L are input to the dementia progression prediction model 41. The dementia progression prediction model 41 outputs a progression prediction result for learning 64L and a score prediction result for learning 50L for the target input data for learning 16L and the clinical trial period for learning 17L.

A loss calculation of the dementia progression prediction model 41 using a cross-entropy function is performed based on the progression prediction result for learning 64L and the correct answer diagnosis result for learning 64CA. A result of the loss calculation is hereinafter referred to as a loss L1. In addition, a loss calculation of the dementia progression prediction model 41 using a regression loss function such as a mean squared error is performed based on the score prediction result for learning 50L and the correct answer score for learning 50CA. A result of the loss calculation is hereinafter referred to as a loss L2.

Various coefficients of the dementia progression prediction model 41 are set to be updated according to the losses L1 and L2, and the dementia progression prediction model 41 is updated according to the update settings. The update setting is performed based on a total loss L represented by Equation (1) below. Note that a is a weight.

L=L1×α+L2×(1−α) (1)
That is, the total loss L is a weighted sum of the loss L1 and the loss L2. α is, for example, 0.5.

In the learning phase, the series of processes of an input of the target input data for learning 16L and the clinical trial period for learning 17L to the dementia progression prediction model 41, an output of the progression prediction result for learning 64L and the score prediction result for learning 50L from the dementia progression prediction model 41, a loss calculation, an update setting, and an update of the dementia progression prediction model 41 are repeatedly performed while the supervised training data 70 is exchanged at least twice. The repetition of the series of processes is ended in a case where prediction accuracy of the progression prediction result for learning 64L and the score prediction result for learning 50L with respect to the correct answer diagnosis result for learning 64CA and the correct answer score for learning 50CA reaches a predetermined set level. The dementia progression prediction model 41 of which the prediction accuracy reaches the set level in this way is stored in the storage 30, and is used in the prediction unit 47. The learning may be ended in a case where the series of processes is repeated a set number of times, regardless of the prediction accuracy of the progression prediction result for learning 64L and the score prediction result for learning 50L with respect to the correct answer diagnosis result for learning 64CA and the correct answer score for learning 50CA.

Note that, although 0.5 has been described as an example of a, the technology of the present disclosure is not limited thereto. Also, a is not limited to a fixed value, and a may be changed, for example, between the initial period of the learning phase and the other period. For example, in the initial period of the learning phase, a is set to 1, and as the learning progresses, a is gradually decreased, and is eventually set to a fixed value, for example, 0.5.

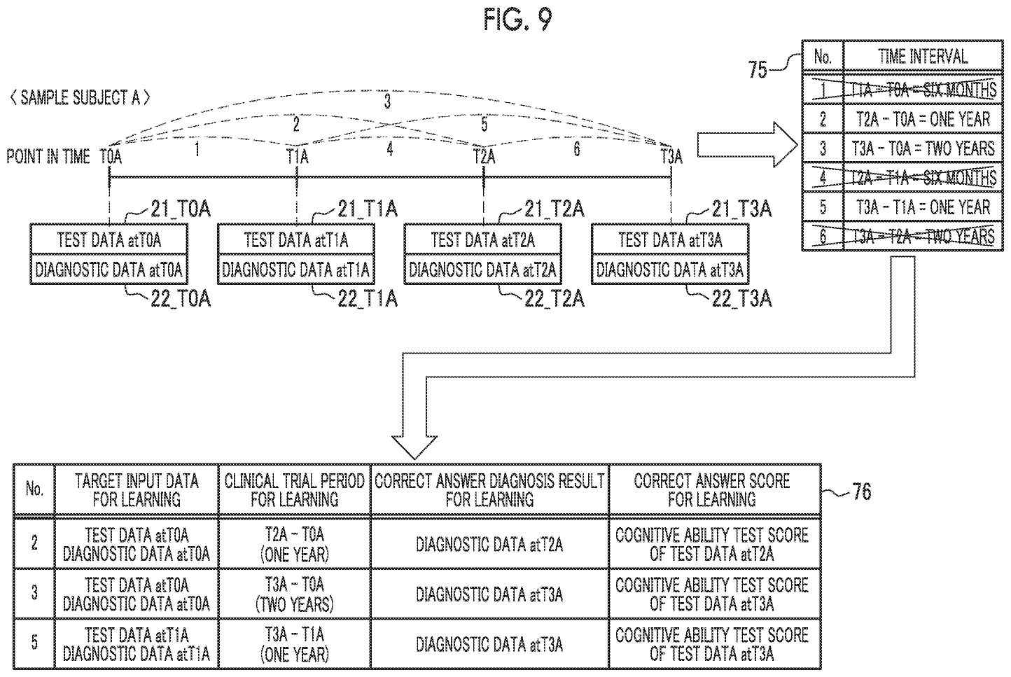

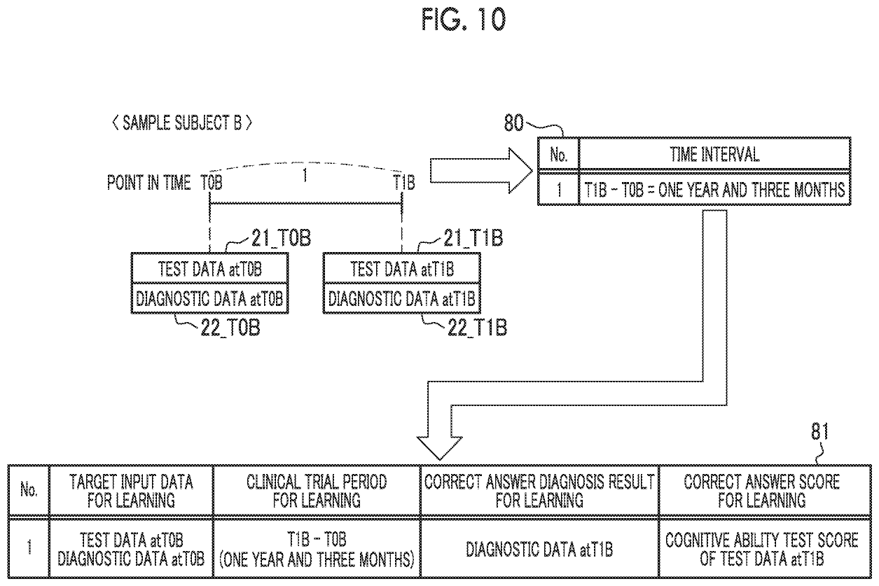

FIGS. 9 and 10 are diagrams for describing the formation of the supervised training data 70. FIG. 9 shows a case of a sample subject A. FIG. 10 shows a case of a sample subject B.

In FIG. 9, the sample subject A has test data 21 and diagnostic data 22 at four points in time of points in time T0A, T1A, T2A, and T3A. Specifically, the sample subject A has test data 21_T0A (denoted as test data atT0A in FIG. 9) and diagnostic data 22_T0A (denoted as diagnostic data atT0A in FIG. 9) at a point in time T0A, test data 21_T1A (denoted as test data atT1A in FIG. 9) and diagnostic data 22_T1A (denoted as diagnostic data atT1A in FIG. 9) at a point in time T1A, test data 21_T2A (denoted as test data atT2A in FIG. 9) and diagnostic data 22_T2A (denoted as diagnostic data atT2A in FIG. 9) at a point in time T2A, and test data 21_T3A (denoted as test data atT3A in FIG. 9) and diagnostic data 22_T3A (denoted as diagnostic data atT3A in FIG. 9) at a point in time T3A.

Table 75 shows the time interval for each point in time. That is, a time interval T1A-T0A between the point in time T0A and the point in time T1A in No. 1, a time interval T2A-T1A between the point in time T1A and the point in time T2A in No. 4, and a time interval T3A-T2A between the point in time T2A and the point in time T3A in No. 6 are six months. A time interval T2A-T0A between the point in time T0A and the point in time T2A in No. 2 and a time interval T3A-T1A between the point in time T1A and the point in time T3A in No. 5 are one year. A time interval T3A-T0A between the point in time T0A and the point in time T3A in No. 3 is two years. Among these No. 1 to No. 6, No. 2 and No. 5 with a time interval of one year and No. 3 with a time interval of two years satisfy the condition of the clinical trial period for learning 17L of one year to two years.

Therefore, as shown in Table 76, from the sample subject A, it is possible to generate a total of three pieces of supervised training data 70 of No. 2, No. 3, and No. 5. For example, the supervised training data 70 of No. 2 is data related to the point in time T0A and the point in time T2A. The target input data for learning 16L is the test data 21_T0A and the diagnostic data 22_T0A at the point in time T0A. The clinical trial period for learning 17L is one year of the time interval T2A-T0A between the point in time T0A and the point in time T2A. The correct answer diagnosis result for learning 64CA is the diagnostic data 22_T2A at the point in time T2A. The correct answer score for learning 50CA is the cognitive ability test score 25 of the test data 21_T2A at the point in time T2A. In this case, the point in time T0A corresponds to the start point in time of the clinical trial period for learning 17L, and the point in time T2A corresponds to the end point in time of the clinical trial period for learning 17L.

In addition, for example, the supervised training data 70 of No. 5 is data related to the point in time T1A and the point in time T3A. The target input data for learning 16L is the test data 21_T1A and the diagnostic data 22_T1A at the point in time T1A. The clinical trial period for learning 17L is one year of the time interval T3A-T1A between the point in time T1A and the point in time T3A. The correct answer diagnosis result for learning 64CA is the diagnostic data 22_T3A at the point in time T3A. The correct answer score for learning 50CA is the cognitive ability test score 25 of the test data 21_T3A at the point in time T3A. In this case, the point in time T1A corresponds to the start point in time of the clinical trial period for learning 17L, and the point in time T3A corresponds to the end point in time of the clinical trial period for learning 17L. Note that the numbers of No. 1 to No. 6 correspond to the numbers 1 to 6 of the arcs connecting the points in time on the time axis. The same applies to FIG. 10.

In FIG. 10, the sample subject B has test data 21 and diagnostic data 22 at two points in time of points in time T0B and T1B. Specifically, the sample subject B has test data 21_T0B (denoted as test data atT0B in FIG. 10) and diagnostic data 22_T0B (denoted as diagnostic data atT0B in FIG. 10) at a point in time T0B and test data 21_T1B (denoted as test data atT1B in FIG. 10) and diagnostic data 22_T1B (denoted as diagnostic data atT1B in FIG. 10) at a point in time T1B.

Table 80 shows the time interval between the point in time T0B and the point in time T1B. That is, a time interval T1B-T0B between the point in time T0B and the point in time T1B is one year and three months. This one year and three months satisfies one year to two years that is the condition of the clinical trial period for learning 17L. Therefore, as shown in Table 81, from the sample subject B, it is possible to generate one piece of supervised training data 70 of No. 1. That is, the supervised training data 70 of No. 1 is data related to the point in time T0B and the point in time T1B. The target input data for learning 16L is the test data 21_T0B and the diagnostic data 22_T0B at the point in time T0B. The clinical trial period for learning 17L is one year and three months of the time interval T1B-T0B between the point in time T0B and the point in time T1B. The correct answer diagnosis result for learning 64CA is the diagnostic data 22_T1B at the point in time T1B. The correct answer score for learning 50CA is the cognitive ability test score 25 of the test data 21_T1B at the point in time T1B. In this case, the point in time T0B corresponds to the start point in time of the clinical trial period for learning 17L, and the point in time T1B corresponds to the end point in time of the clinical trial period for learning 17L. In this way, the supervised training data 70 includes the test data 21 and the diagnostic data 22 at two points in time and the interval between the two points in time out of the test data 21 and the diagnostic data 22 at two or more points in time of the same sample subject. Although not shown, for example, in a case of a sample subject having the test data 21 and the diagnostic data 22 at six points in time, ₆C₂=6×5÷2=15, and thus, of the 15 pieces of data, it is possible to generate the supervised training data 70 with the data that satisfies the conditions of the clinical trial period for learning 17L. Further, for example, in a case of a sample subject having the test data 21 and the diagnostic data 22 at eight points in time, ₈C₂=8×7÷2=28, and thus, of the 28 pieces of data, it is possible to generate the supervised training data 70 with the data that satisfies the conditions of the clinical trial period for learning 17L.

The supervised training data 70 is not limited to data including the input data related to dementia at two or more points in time of the same sample subject and the time intervals thereof. The input data related to dementia of a plurality of sample subjects having the same and/or similar dementia symptoms and time intervals thereof may be combined to generate the input data related to dementia at two or more points in time and time intervals thereof, which may be used as the supervised training data 70. Examples of the sample subject having the same and/or similar dementia symptoms include the sample subject having the same and/or similar test data 21 and/or the diagnostic data 22. In addition, the input data related to dementia of a plurality of sample subjects having the same and/or similar attributes and time intervals thereof may be combined to generate the input data related to dementia at two or more points in time and time intervals thereof, which may be used as the supervised training data 70. Examples of the sample subject having the same and/or similar attributes include the sample subject having the same and/or similar age 23 and/or the gender 24. The input data related to dementia of a plurality of sample subjects having the same and/or similar dementia symptoms and having the same and/or similar attributes and time intervals thereof may be combined to generate the input data related to dementia at two or more points in time and time intervals thereof, which may be used as the supervised training data 70.

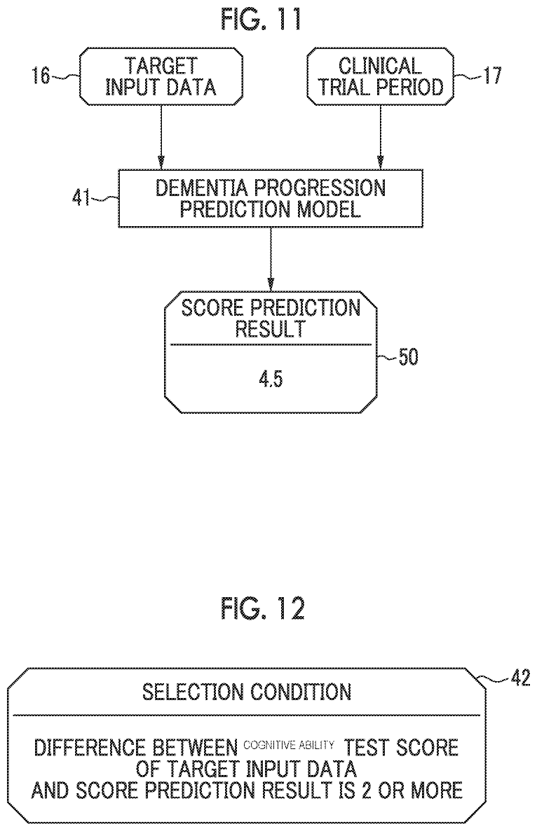

As shown in FIG. 11 as an example, the prediction unit 47 inputs the target input data 16 and the clinical trial period 17 to the dementia progression prediction model 41, and causes the dementia progression prediction model 41 to output a score prediction result 50. The progression prediction result 64 is also output from the dementia progression prediction model 41, but the prediction unit 47 discards the progression prediction result 64 and outputs only the score prediction result 50 to the determination unit 48. FIG. 11 exemplifies a case where the score prediction result 50 is 4.5.

As shown in FIG. 12 as an example, the selection condition 42 is a content that a difference between the cognitive ability test score 25 of the target input data 16 and the score prediction result 50 is 2 or more. In a case where the difference between the cognitive ability test score 25 of the target input data 16 and the score prediction result 50 is 2 or more, the progression of Alzheimer's dementia is relatively rapid. Therefore, the subject candidate in which the difference between the cognitive ability test score 25 of the target input data 16 and the score prediction result 50 is 2 or more is suitable as a subject for the clinical trial. On the contrary, whether the subject candidate in which the difference between the cognitive ability test score 25 of the target input data 16 and the score prediction result 50 is less than 2 is not suitable as a subject for the clinical trial because it is not clear whether the progression is being suppressed by the efficacy of anti-dementia drugs or the progression is being delayed due to reasons specific to the subject.

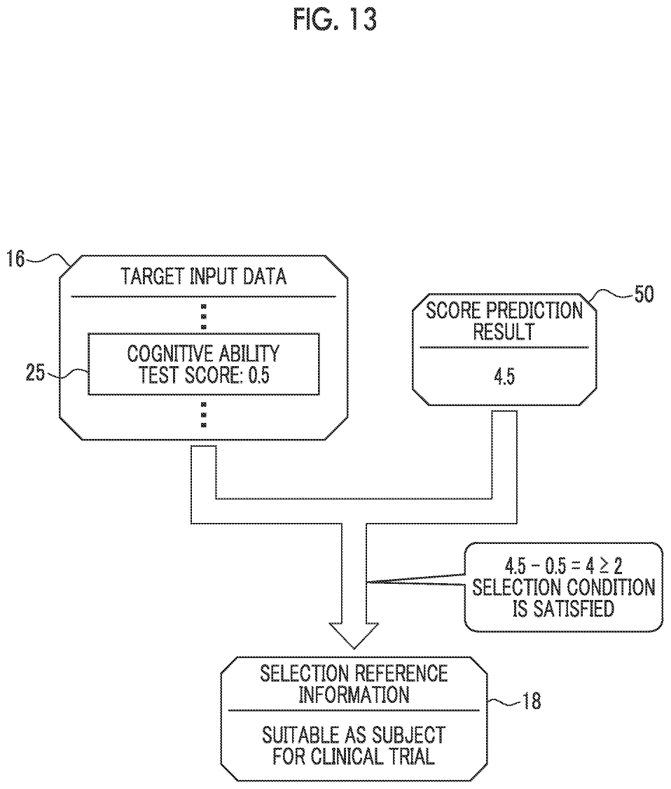

Therefore, as shown in FIG. 13 as an example, in a case where the difference between the cognitive ability test score 25 of the target input data 16 and the score prediction result 50 is 2 or more and the selection condition 42 is satisfied, the determination unit 48 generates selection reference information 18 having a content that the subject candidate is suitable as a subject for the clinical trial. FIG. 13 exemplifies a case where the cognitive ability test score 25 of the target input data 16 is 0.5, the score prediction result 50 is 4.5, and the difference is 4, which is 2 or more.

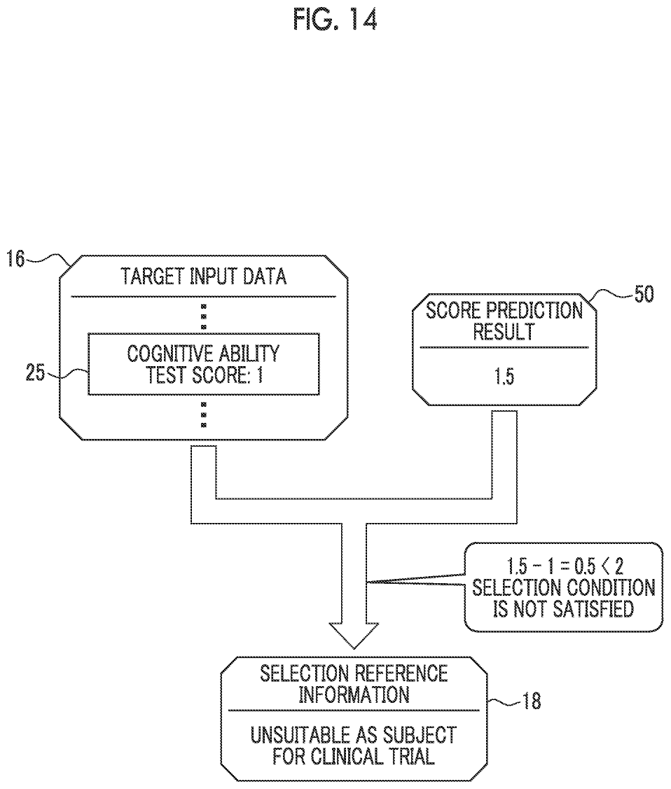

On the other hand, as shown in FIG. 14 as an example, in a case where the difference between the cognitive ability test score 25 of the target input data 16 and the score prediction result 50 is less than 2 and the selection condition 42 is not satisfied, the determination unit 48 generates selection reference information 18 having a content that the subject candidate is unsuitable as a subject for the clinical trial. FIG. 14 exemplifies a case where the cognitive ability test score 25 of the target input data 16 is 1, the score prediction result 50 is 1.5, and the difference is 0.5, which is less than 2.

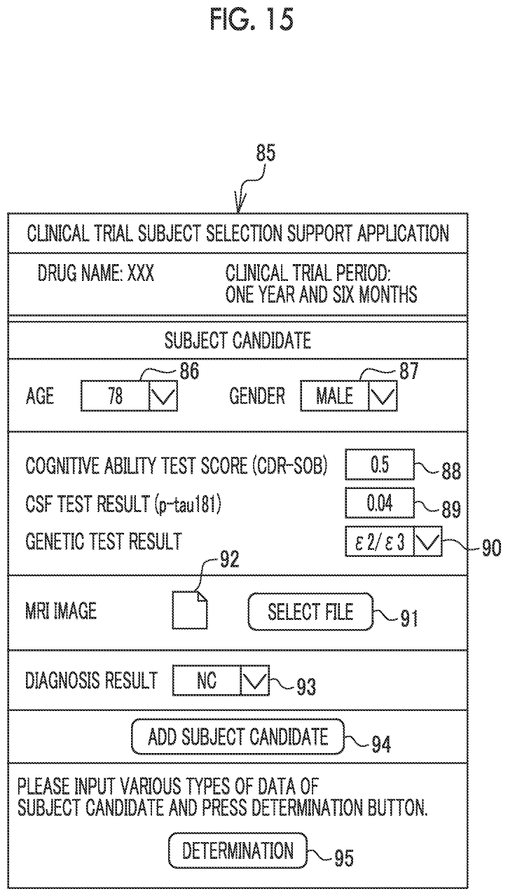

FIG. 15 shows an example of a clinical trial subject selection support screen 85 displayed on the display 13 of the user terminal 11. On the clinical trial subject selection support screen 85, a pull-down menu 86 for selecting the age 23 of the subject candidate, a pull-down menu 87 for selecting the gender 24 of the subject candidate, an input box 88 for the cognitive ability test score 25, an input box 89 for the CSF test result 26, and a pull-down menu 90 for selecting the genetic test result 27 are provided.

On the clinical trial subject selection support screen 85, a file selection button 91 for selecting a file of the MRI image 28 is provided. In a case where the file of the MM image 28 is selected, a file icon 92 is displayed next to the file selection button 91. The file icon 92 is not displayed in a case where a file is not selected. Further, a pull-down menu 93 for selecting the diagnosis result (diagnostic data 22) is provided on the clinical trial subject selection support screen 85.

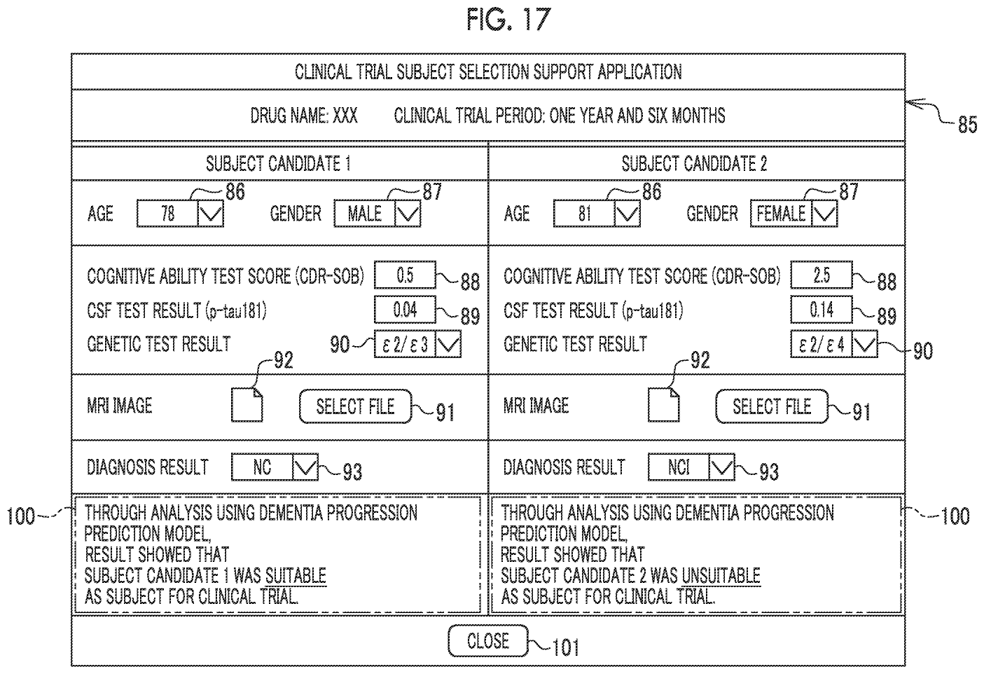

A subject candidate addition button 94 is provided on the clinical trial subject selection support screen 85. In a case where the subject candidate addition button 94 is selected, a set of the pull-down menus 86, 87, 90, and 93, the input boxes 88 and 89, and the file selection button 91 is added to the clinical trial subject selection support screen 85 (refer to FIG. 17). The subject candidate addition button 94 can be selected a plurality of times. Accordingly, it is possible to input the target input data 16 of two or more subject candidates on one clinical trial subject selection support screen 85.

A determination button 95 is disposed at the bottom of the clinical trial subject selection support screen 85. In a case where the determination button 95 is selected, the distribution request 15 including the target input data 16 and the clinical trial period 17 is transmitted from the user terminal 11 to the clinical trial subject selection support server 10. The target input data 16 is composed of the contents selected by the pull-down menus 86, 87, 90, and 93, the contents input in the input boxes 88 and 89, and the MRI image 28 selected by the file selection button 91.

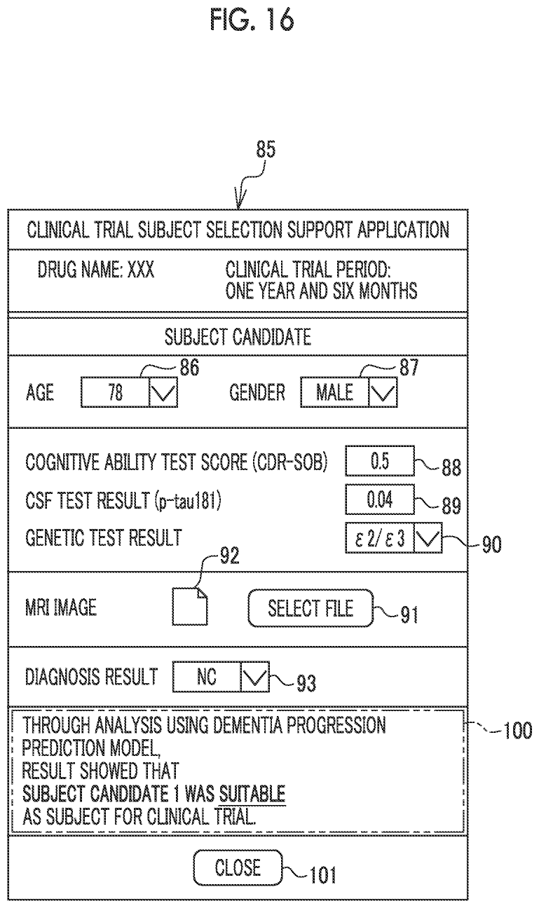

In a case where the selection reference information 18 from the clinical trial subject selection support server 10 is received, the clinical trial subject selection support screen 85 transitions as shown in FIG. 16 as an example. Specifically, a message 100 indicating the selection reference information 18 is displayed. FIG. 16 exemplifies a case where the selection reference information 18 has a content that the subject candidate is suitable as a subject for the clinical trial. The display of the clinical trial subject selection support screen 85 disappears by selecting a close button 101.

The clinical trial subject selection support screen 85 in a case where the subject candidate addition button 94 is selected and the subject candidate is added is as shown in FIG. 17 as an example. FIG. 17 shows an example in which the message 100 indicating the selection reference information 18 of two subject candidates is displayed.

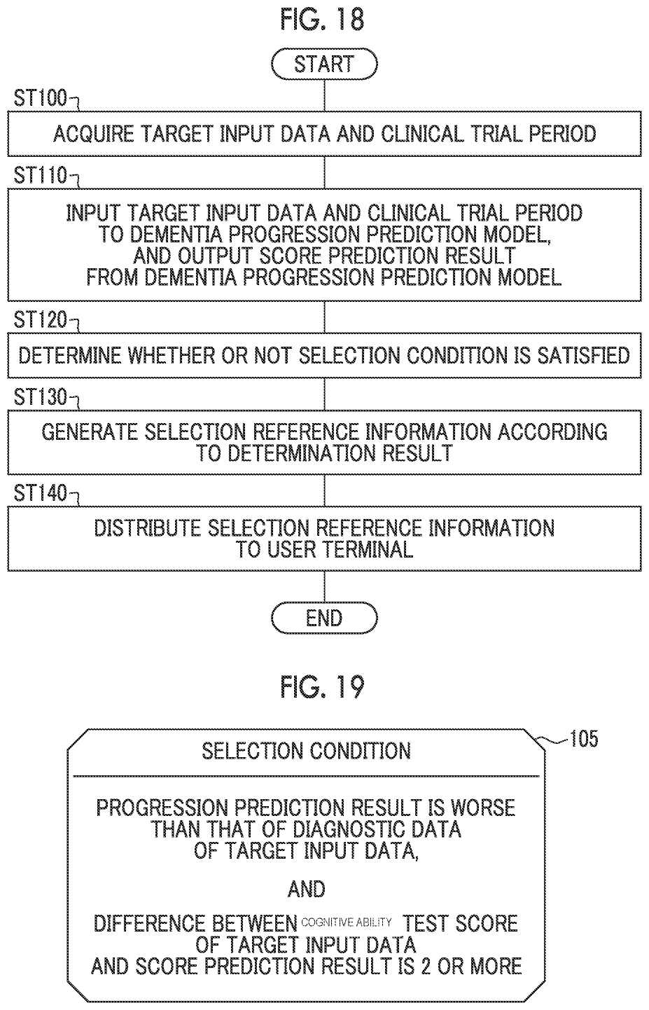

Next, an operation according to the above configuration will be described with reference to a flowchart shown in FIG. 18. First, in a case where the operation program 40 is activated in the clinical trial subject selection support server 10, as shown in FIG. 6, the CPU 32 of the clinical trial subject selection support server 10 functions as the reception unit 45, the RW control unit 46, the prediction unit 47, the determination unit 48, and the distribution control unit 49.

First, in the reception unit 45, the distribution request 15 from the user terminal 11 is received, and thus the target input data 16 and the clinical trial period 17 are acquired (Step ST100). The target input data 16 and the clinical trial period 17 are output from the reception unit 45 to the prediction unit 47.

As shown in FIG. 11, in the prediction unit 47, the target input data 16 and the clinical trial period 17 are input to the dementia progression prediction model 41, and the score prediction result 50 is output from the dementia progression prediction model 41 (Step ST110). The score prediction result 50 is output from the prediction unit 47 to the determination unit 48.

As shown in FIGS. 13 and 14, in the determination unit 48, the difference between the cognitive ability test score 25 of the target input data 16 and the score prediction result 50 is calculated. Then, it is determined whether the difference is 2 or more and thus the selection condition 42 is satisfied or the difference is less than 2 and the selection condition 42 is not satisfied (Step ST120). In a case where the selection condition 42 is satisfied, the selection reference information 18 having a content that the subject candidate is suitable as a subject for the clinical trial is generated by the determination unit 48, as shown in FIG. 13 (Step ST130). On the other hand, in a case where the selection condition 42 is not satisfied, the selection reference information 18 having a content that the subject candidate is unsuitable as a subject for the clinical trial is generated by the determination unit 48, as shown in FIG. 14 (Step ST130).

The selection reference information 18 is output from the determination unit 48 to the distribution control unit 49. The selection reference information 18 is distributed to the user terminal 11 that is the transmission source of the distribution request 15 under the control of the distribution control unit 49 (Step ST140).

As described above, the CPU 32 of the clinical trial subject selection support server 10 comprises the reception unit 45, the prediction unit 47, and the determination unit 48. The reception unit 45 receives the distribution request 15 to acquire the target input data 16 which is input data related to dementia of the subject candidate for the clinical trial of the anti-dementia drug, and the clinical trial period 17 of the anti-dementia drug. The prediction unit 47 inputs the target input data 16 and the clinical trial period 17 of the anti-dementia drug to the dementia progression prediction model 41, and causes the dementia progression prediction model 41 to output the score prediction result 50 which is the prediction result regarding dementia of the subject candidate at the end point in time of the clinical trial period 17. The determination unit 48 outputs the selection reference information 18 for determining whether or not to select the subject candidate as the subject for the clinical trial, according to the score prediction result 50.

As shown in FIGS. 8 to 10, the dementia progression prediction model 41 is trained using the supervised training data 70 including the accumulated target input data for learning 16L related to dementia at two or more points in time and the clinical trial period for learning 17L. Since the clinical trial period for learning 17L is included as the time interval of the input data, It is possible to improve the prediction accuracy of the score prediction result 50 as compared with the method of Document 1 in which test data of three or more points in time are provided as a set of supervised training data to the RNN for learning. Since more supervised training data 70 can be prepared than in the method of Document 1, overlearning can be prevented. Therefore, it is possible to suppress a decrease in accuracy of predicting the progression of dementia, and it is thus possible to improve the accuracy of predicting the progression of dementia. As a result, it is possible to select a person suitable as a subject for a clinical trial of an anti-dementia drug with high accuracy.

The clinical trial period for learning 17L is an interval set according to the clinical trial period 17. Therefore, the dementia progression prediction model 41 can be a machine learning model specialized for prediction at the time interval corresponding to the clinical trial period 17, and it is possible to further improve the selection accuracy of a person suitable as a subject for a clinical trial.

The input data includes the test data 21 indicating a result of a test related to the dementia and the diagnostic data 22 indicating a result of the diagnosis related to the dementia. Therefore, it is possible to contribute to improving the prediction accuracy of the score prediction result 50. Note that the input data may include at least one of the test data 21 or the diagnostic data 22.

The dementia progression prediction model 41 outputs, as a prediction result, the score prediction result 50 that is a prediction result of a score quantitatively representing a degree of progression of dementia. Therefore, it is possible to follow the related-art method of selecting a person to participate in a clinical trial using the cognitive ability test score 25.

The dementia progression prediction model 41 further outputs, as a prediction result, the progression prediction result 64 that is a prediction result of a class qualitatively representing a degree of progression of dementia. Compared to predicting only the cognitive ability test score 25 which is a continuous amount and has a certain range, it is possible to improve the prediction accuracy of the score prediction result 50 by making predictions together with the progression prediction result 64, which is relatively simple with several cases (in this example, four types of normal control/preclinical AD/mild cognitive impairment/Alzheimer's dementia).

The clinical trial period 17 may not be included in the distribution request 15. Since the clinical trial period 17 is known in advance, the clinical trial period 17 may be stored in the storage 30. In this case, the clinical trial period 17 is acquired by the RW control unit 46 reading out the clinical trial period 17 from the storage 30. The RW control unit 46 outputs the read-out clinical trial period 17 to the prediction unit 47.

For example, a plurality of types of dementia progression prediction models 41 according to different clinical trial periods 17, such as a dementia progression prediction model 41 with the clinical trial period 17 of one year, a dementia progression prediction model 41 with the clinical trial period 17 two years, may be prepared.

The selection reference information is not limited to the selection reference information 18 having the content that the exemplary subject candidate is suitable/unsuitable as a subject for the clinical trial. The score prediction result 50 and/or the progression prediction result 64 itself may be distributed to the user terminal 11 as the selection reference information. In this case, determination regarding whether or not the subject candidate is suitable as a subject for the clinical trial is performed by the drug discovery staff with reference to the score prediction result 50 and/or the progression prediction result 64. In this case, the selection condition 42 is not necessary.

A selection condition 105 shown in FIG. 19 as an example may be used. The selection condition 105 is a content that the progression prediction result 64 is worse than that of the diagnostic data 22 of the target input data 16 and that the difference between the cognitive ability test score 25 of the target input data 16 and the score prediction result 50 is 2 or more. The fact that the progression prediction result 64 is worse than that of the diagnostic data 22 of the target input data 16 means a case where the diagnostic data 22 of the target input data 16 is normal control and the progression prediction result 64 is a preclinical AD, mild cognitive impairment, or Alzheimer's dementia and a case where the diagnostic data 22 of the target input data 16 is mild cognitive impairment and the progression prediction result 64 is Alzheimer's dementia. Further, the fact that the progression prediction result 64 is worse than that of the diagnostic data 22 of the target input data 16 means a case where the diagnostic data 22 of the target input data 16 is the preclinical AD and the progression prediction result 64 is mild cognitive impairment or Alzheimer's dementia. In this way, the selection condition may be a content including the progression prediction result 64 instead of or in addition to the score prediction result 50.

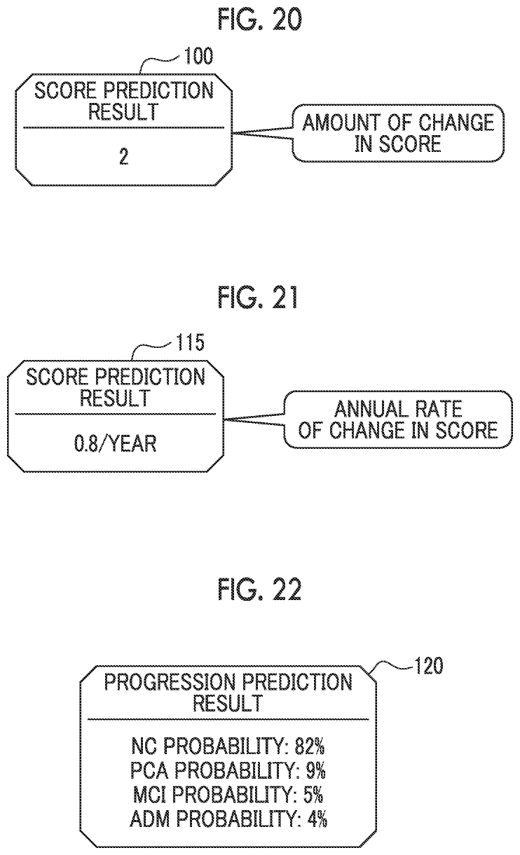

The score prediction result is not limited to the score prediction result 50 indicating the cognitive ability test score 25 itself of the first embodiment. A score prediction result 110 shown in FIG. 20 as an example and a score prediction result 115 shown in FIG. 21 as an example may be used.

The score prediction result 110 shown in FIG. 20 indicates the amount of change in the cognitive ability test score 25. By adding this amount of change to the cognitive ability test score 25 of the target input data 16 input to the dementia progression prediction model 41 or subtracting this amount of change from the cognitive ability test score 25, the cognitive ability test score 25 at the end point in time of the clinical trial period 17 can be calculated. In FIG. 20, 2 is exemplified as the amount of change. Therefore, by adding 2 to the cognitive ability test score 25 of the target input data 16 input to the dementia progression prediction model 41, the cognitive ability test score 25 at the end point in time of the clinical trial period 17 is calculated.

The score prediction result 115 shown in FIG. 21 indicates an annual rate of change in the cognitive ability test score 25. The annual rate of change is a rate that indicates how much the cognitive ability test score 25 changes in one year. By multiplying this amount of change by the clinical trial period 17 and adding the multiplication result to the cognitive ability test score 25 of the target input data 16 input to the dementia progression prediction model 41 or subtracting the multiplication result from the cognitive ability test score 25, the cognitive ability test score 25 at the end point in time of the clinical trial period 17 can be calculated. In FIG. 21, 0.8/year is exemplified as the annual rate of change. Therefore, by multiplying 0.8 by the clinical trial period 17 and adding the multiplication result to the cognitive ability test score 25 of the target input data 16 input to the dementia progression prediction model 41, the cognitive ability test score 25 at the end point in time of the clinical trial period 17 is calculated. In a case where the clinical trial period 17 is, for example, one year and six months, the multiplication result is 0.8×1.5=1.2. In addition, in a case where the clinical trial period 17 is, for example, two years, the multiplication result is 0.8×2=1.6.

The progression prediction result is not limited to the progression prediction result 64 having a content of any one of normal control, preclinical AD, mild cognitive impairment, or Alzheimer's dementia as exemplified. As in a progression prediction result 120 shown in FIG. 22 as an example, a probability of each of normal control, preclinical AD, mild cognitive impairment, and Alzheimer's dementia may be used.

Further, the progression prediction result is not limited to Alzheimer's dementia, and more generally, the progression prediction result may be a content that a subject candidate is any one of normal control, preclinical AD, mild cognitive impairment, or dementia. Subjective cognitive impairment (SCI) and/or subjective cognitive decline (SCD) may be added as a prediction target. In addition, the progression prediction result may include a content that the subject candidate develops Alzheimer's dementia two years later or does not develop Alzheimer's dementia two years later. In addition, for example, the progression prediction result may include a content that a degree of progression of the subject candidate to dementia three years later is fast or slow. Further, the progression prediction result may include a content indicating whether the subject candidate progresses to MCI from normal control or preclinical AD or whether the subject candidate progresses to Alzheimer's dementia from normal control, preclinical AD, or MCI.

### Second Embodiment

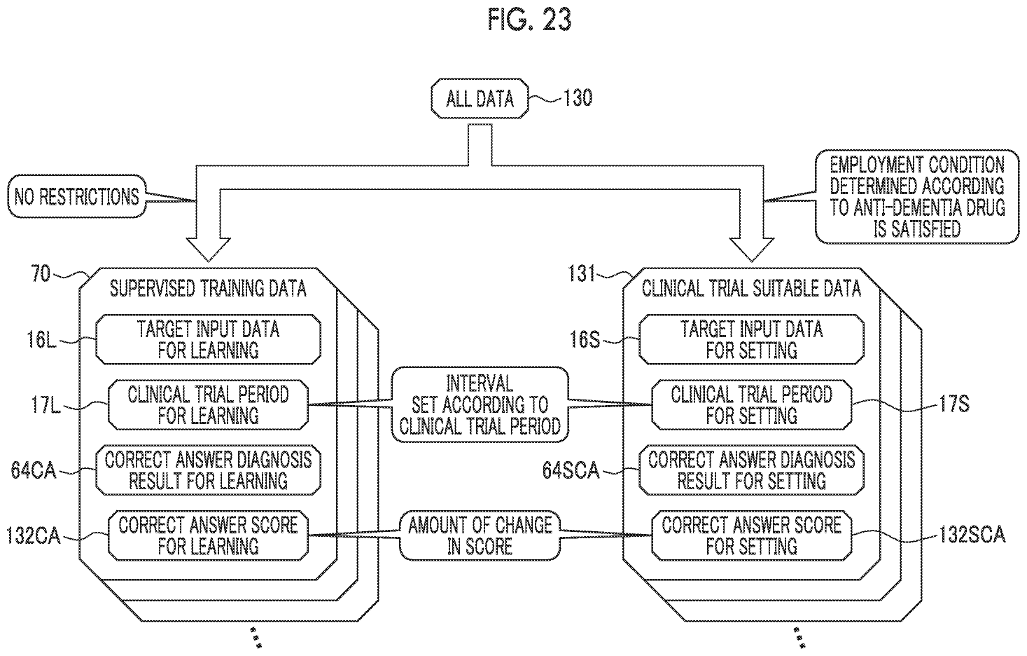

As shown in FIG. 23 as an example, in a second embodiment, clinical trial suitable data 131 is generated from all data 130, in addition to the supervised training data 70. While the supervised training data 70 has no restrictions, the clinical trial suitable data 131 has the restriction that it satisfies employment conditions. The employment conditions are determined in advance according to the anti-dementia drug, and include, for example, a person who is 65 years old or older and has a mini-mental state examination (MMSE) score of 25 points or less. Therefore, the clinical trial suitable data 131 has a smaller number of data items than the supervised training data 70.

The clinical trial suitable data 131 is a set of target input data for setting 16S, a clinical trial period for setting 17S, a correct answer diagnosis result for setting 64SCA, and a correct answer score for setting 132SCA. The target input data for setting 16S corresponds to the target input data for learning 16L of the supervised training data 70, and the clinical trial period for setting 17S corresponds to the clinical trial period for learning 17L of the supervised training data 70. The clinical trial period for setting 17S is an interval set according to the clinical trial period 17, as in the clinical trial period for learning 17L. In a case where the clinical trial period 17 is, for example, one year and six months, the clinical trial period for setting 17S is one year to two years plus or minus six months to one year and six months. The target input data for setting 16S is an example of “input data of clinical trial suitable data” according to the technology of the present disclosure. Further, the clinical trial period for setting 17S is an example of a “time interval of clinical trial suitable data” according to the technology of the present disclosure.

The correct answer diagnosis result for setting 64SCA corresponds to the correct answer diagnosis result for learning 64CA of the supervised training data 70, and the correct answer score for setting 132SCA corresponds to a correct answer score for learning 132CA of the supervised training data 70. The correct answer score for learning 132CA and the correct answer score for setting 132SCA are annual rates of change (hereinafter simply referred to as amounts of change) in the cognitive ability test scores 25 shown in FIG. 21. The correct answer score for setting 132SCA is an example of “correct answer data included in the clinical trial suitable data” according to the technology of the present disclosure.

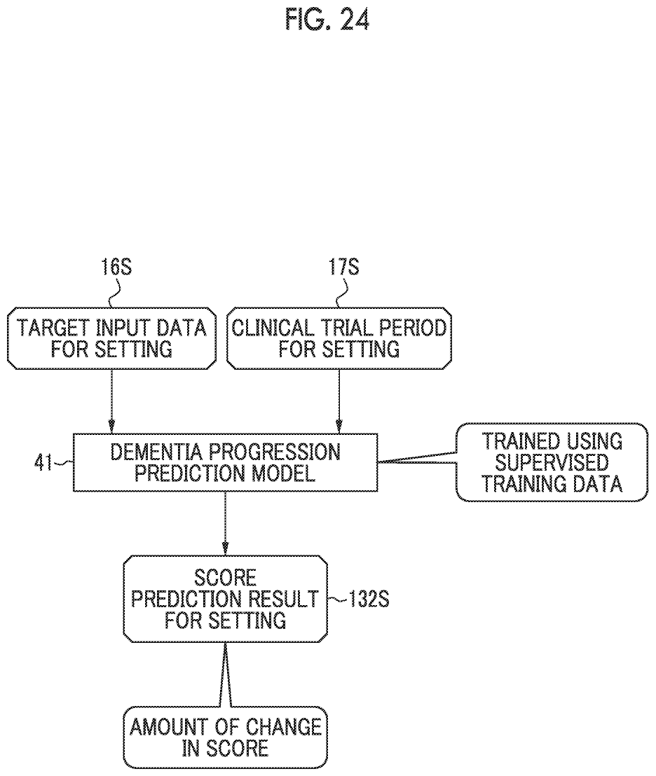

As shown in FIG. 24 as an example, the target input data for setting 16S of the clinical trial suitable data 131 and the clinical trial period for setting 17S are input to the dementia progression prediction model 41 trained using the supervised training data 70. Accordingly, a score prediction result for setting 132S is output from the dementia progression prediction model 41. The score prediction result for setting 132S is an amount of change in the cognitive ability test score 25, as in the correct answer score for setting 132SCA. The score prediction result for setting 132S is an example of a “prediction result for setting” according to the technology of the present disclosure.

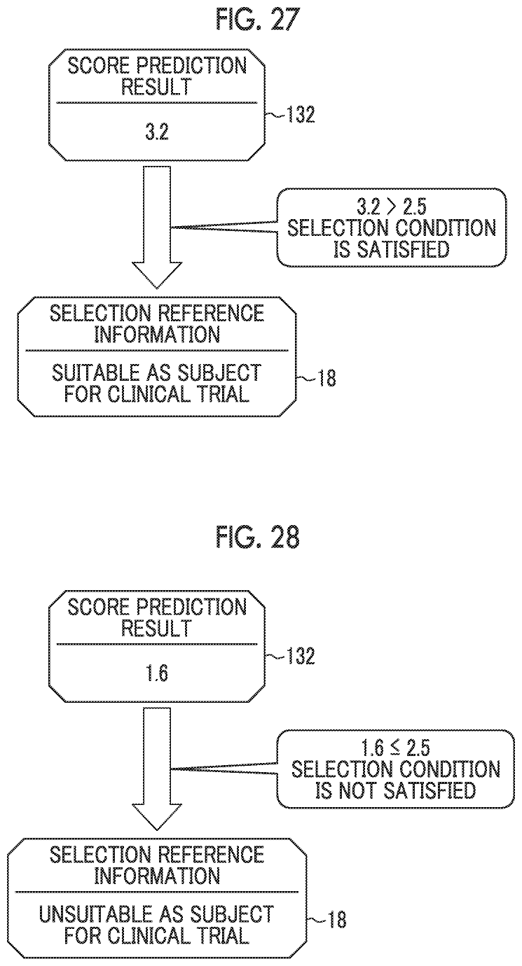

As described above, since the supervised training data 70 is data generated without any restriction, there is a large amount of data that does not satisfy the employment condition. Therefore, some error occurs in the score prediction result for setting 132S obtained by inputting the target input data for setting 16S of the clinical trial suitable data 131 and the clinical trial period for setting 17S to the dementia progression prediction model 41 trained using the supervised training data 70. This error may also occur in a score prediction result 132 (refer to FIG. 27) output by inputting the target input data 16 of the subject candidate and the clinical trial period 17 to the dementia progression prediction model 41. Therefore, in a case where the selection conditions are determined without correcting this error, a person suitable as a subject for the clinical trial may be omitted from the selection, or conversely, a person unsuitable as a subject for the clinical trial may be selected. Therefore, a method of correcting the above error will be described below.

<Method 1>

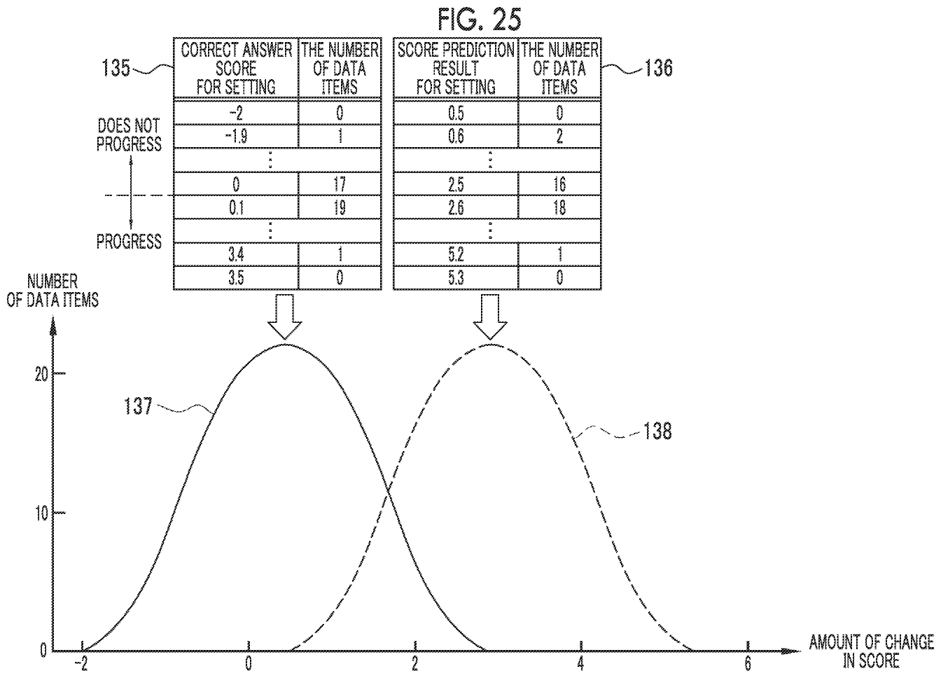

As shown in FIG. 25 as an example, Table 135 summarizes the number of data items of the clinical trial suitable data 131 for each correct answer score for setting 132SCA in increments of 0.1. Similarly, Table 136 summarizes the number of data items of the clinical trial suitable data 131 for each score prediction result for setting 132S in increments of 0.1. From Table 135, it is possible to generate a correct answer score distribution for setting 137, which is the distribution of the number of data items of the correct answer score for setting 132SCA. Further, from Table 136, it is possible to generate a score prediction result distribution for setting 138, which is the distribution of the number of data items of the score prediction result for setting 132S. The correct answer score distribution for setting 137 is an example of a “correct answer data distribution” according to the technology of the present disclosure. Further, the score prediction result distribution for setting 138 is an example of a “prediction result distribution for setting” according to the technology of the present disclosure. As can be seen from the correct answer score distribution for setting 137 and the score prediction result distribution for setting 138, an error occurs between the correct answer score for setting 132SCA and the score prediction result for setting 132S. Note that, here, for convenience of description, the error is drawn in an exaggerated manner.

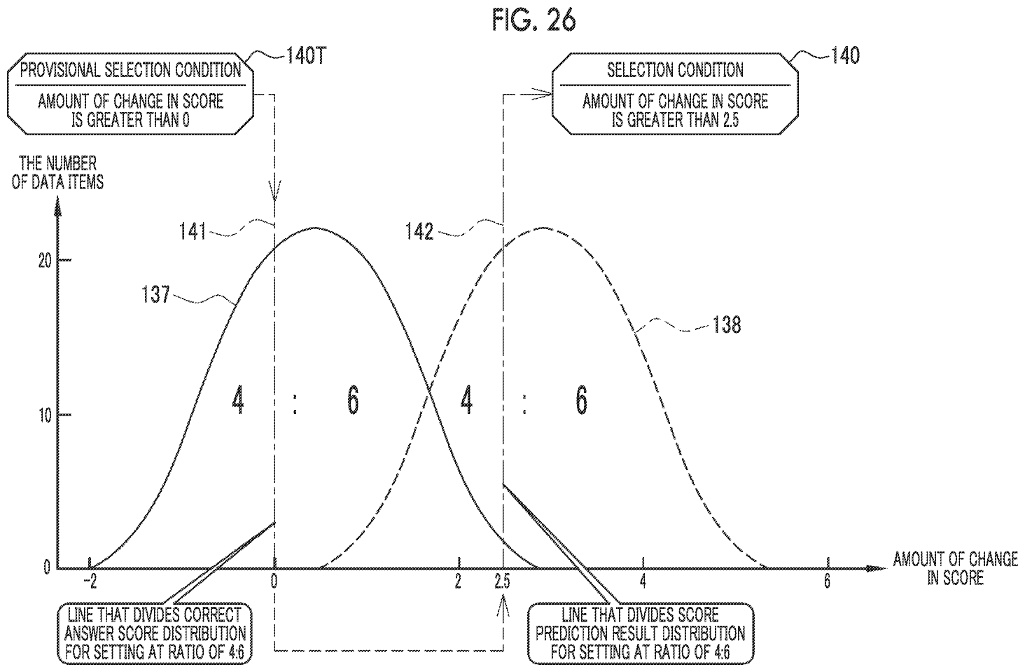

As shown in FIG. 26 as an example, in Method 1, first, a provisional selection condition 140T is set for the correct answer score distribution for setting 137. Next, the provisional selection condition 140T is set to a selection condition 140 by applying the provisional selection condition 140T to the score prediction result distribution for setting 138. Specifically, the amount of change indicated by a line 142 that divides the score prediction result distribution for setting 138 at the same ratio as the ratio at which a line 141 drawn on the amount of change in the cognitive ability test score 25 included in the provisional selection condition 140T divides the correct answer score distribution for setting 137 is set as the selection condition 140.

FIG. 26 exemplifies a case where the provisional selection condition 140T that the amount of change in the cognitive ability test score 25 is greater than 0 is set. Here, a person whose amount of change is greater than 0 is a person whose dementia has progressed when the clinical trial period 17 has elapsed. On the contrary, a person whose amount of change is 0 or less is a person whose dementia has not progressed when the clinical trial period 17 has elapsed.

FIG. 26 exemplifies a case where the line 141 drawn to the amount of change of 0 is a line that divides the correct answer score distribution for setting 137 at a ratio of 4:6. In this case, an amount of change of 2.5 indicated by a line 142 that divides the score prediction result distribution for setting 138 at a ratio of 4:6, which follows the line 141, is set as the selection condition 140. That is, the selection condition 140 has a content that the amount of change in the cognitive ability test score 25 is greater than 2.5.

As shown in FIG. 27 as an example, in a case where the score prediction result 132 obtained by inputting the target input data 16 of the subject candidate and the clinical trial period 17 to the dementia progression prediction model 41 satisfies the selection condition 140, the determination unit 48 generates the selection reference information 18 having a content that the subject candidate is suitable as a subject for the clinical trial. FIG. 27 exemplifies a case where the score prediction result 132 is 3.2.

On the other hand, as shown in FIG. 28 as an example, in a case where the score prediction result 132 obtained by inputting the target input data 16 of the subject candidate and the clinical trial period 17 to the dementia progression prediction model 41 does not satisfy the selection condition 140, the determination unit 48 generates the selection reference information 18 having a content that the subject candidate is unsuitable as a subject for the clinical trial. FIG. 28 exemplifies a case where the score prediction result 132 is 1.6.

As described above, in Method 1, the selection condition 140 is set based on the correct answer score distribution for setting 137, which is the distribution of the number of data items of the correct answer score for setting 132SCA included in the clinical trial suitable data 131, and the score prediction result distribution for setting 138, which is the distribution of the number of data items of the score prediction result for setting 132S. More specifically, the selection condition 140 is set by applying the provisional selection condition 140T set in the correct answer score distribution for setting 137 to the score prediction result distribution for setting 138. Therefore, it is possible to correct an error that occurs in the score prediction result 132. It is possible to greatly reduce the probability of omitting a person suitable as a subject for the clinical trial from the selection, or conversely, selecting a person unsuitable as a subject for the clinical trial.

<Method 2>

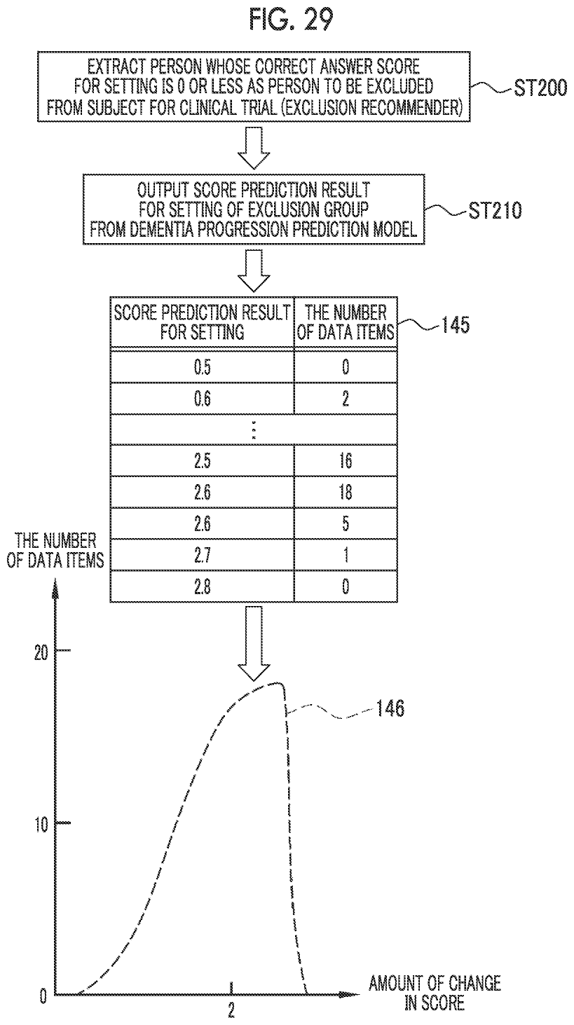

As shown in FIG. 29 as an example, in Method 2, first, as shown in Step ST200, an exclusion group, which is a group of persons to be excluded from the subject for the clinical trial (hereinafter abbreviated as exclusion recommenders), is extracted based on the correct answer score for setting 132SCA. FIG. 29 exemplifies a case where a person whose correct answer score for setting 132SCA is 0 or less is extracted as an exclusion recommender. Next, as shown in Step ST210, the target input data for setting 16S and the clinical trial period for setting 17S of the clinical trial suitable data 131 of the exclusion recommender are input to the dementia progression prediction model 41 trained using the supervised training data 70 and the score prediction result for setting 132S is output from the dementia progression prediction model 41.

Table 145 summarizes the number of data items of the clinical trial suitable data 131 for each score prediction result for setting 132S output in Step ST210. From this Table 145, it is possible to generate a score prediction result distribution for exclusion group setting 146, which is the distribution of the number of data items of the score prediction result for setting 132S of the exclusion group. The score prediction result distribution for exclusion group setting 146 is an example of an “exclusion group prediction result distribution” according to the technology of the present disclosure.

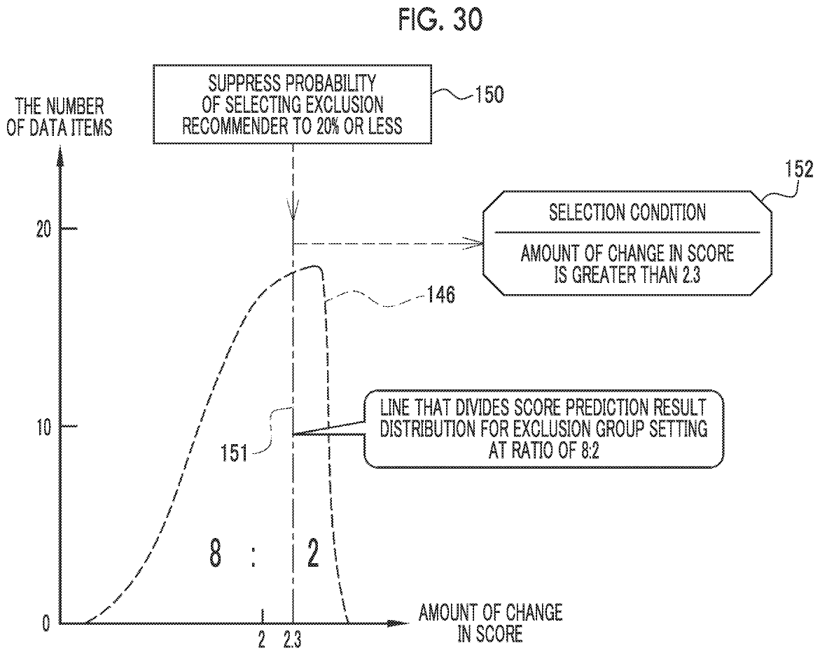

As shown in FIG. 30 as an example, in Method 2, a policy 150 is established by the user as to how many exclusion recommenders are allowed to be selected as subjects for the clinical trial. Then, the amount of change indicated by a line 151 according to the policy 150 and drawn with respect to the score prediction result distribution for exclusion group setting 146 is set as a selection condition 152.

FIG. 30 exemplifies a case where the policy 150 of suppressing the probability of selecting an exclusion recommender to 20% or less is established. In this case, the line 151 is a line that divides the score prediction result distribution for exclusion group setting 146 at a ratio of 8:2. The amount of change of 2.3 indicated by this line 151 is set as the selection condition 152. That is, the selection condition 152 has a content that the amount of change in the cognitive ability test score 25 is greater than 2.3.

As described above, in Method 2, the selection condition 152 is set based on the score prediction result distribution for exclusion group setting 146, which is the distribution of the number of data items of the score prediction result for setting 132S of the exclusion group, which is a group to be excluded from the subject for the clinical trial, the group being extracted based on the correct answer score for setting 132SCA included in the clinical trial suitable data 131. Therefore, it is possible to suppress, to a certain extent, a probability of selecting a person who is unsuitable as a subject for the clinical trial, that is, an exclusion recommender. Since the selection conditions 152 looser than Method 1, which greatly reduces the probability of selecting the exclusion recommender, can be set, it is possible to increase the number of persons to be the subjects for the clinical trial as compared with Method 1.

<Method 3>

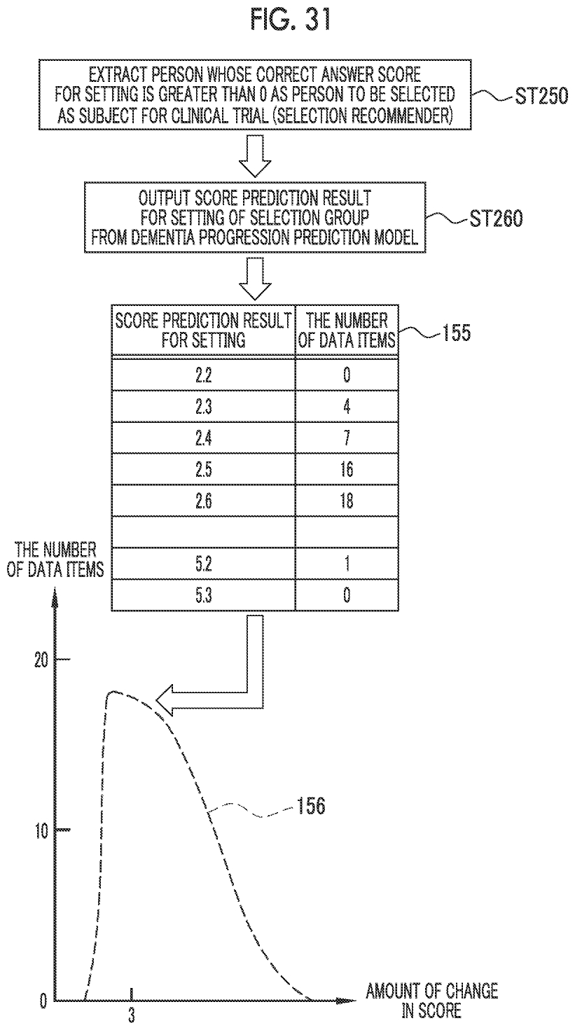

As shown in FIG. 31 as an example, in Method 3, first, as shown in Step ST250, a selection group, which is a group of persons to be selected as the subject for the clinical trial (hereinafter abbreviated as selection recommenders), is extracted based on the correct answer score for setting 132SCA. FIG. 31 exemplifies a case where a person whose correct answer score for setting 132SCA is greater than 0 is extracted as a selection recommender. Next, as shown in Step ST260, the target input data for setting 16S and the clinical trial period for setting 17S of the clinical trial suitable data 131 of the selection recommender are input to the dementia progression prediction model 41 trained using the supervised training data 70 and the score prediction result for setting 132S is output from the dementia progression prediction model 41.

Table 155 summarizes the number of data items of the clinical trial suitable data 131 for each score prediction result for setting 132S output in Step ST260. From this Table 155, it is possible to generate a score prediction result distribution for selection group setting 156, which is the distribution of the number of data items of the score prediction result for setting 132S of the selection group. The score prediction result distribution for selection group setting 156 is an example of a “selection group prediction result distribution” according to the technology of the present disclosure.

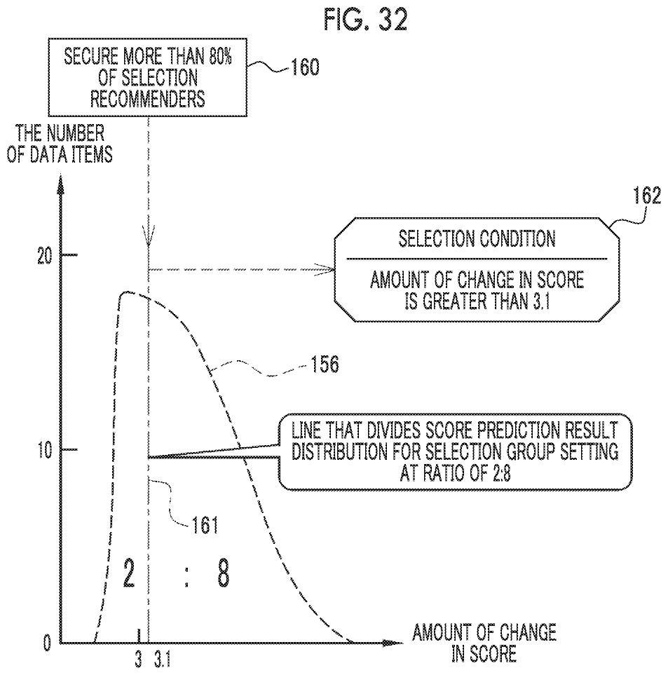

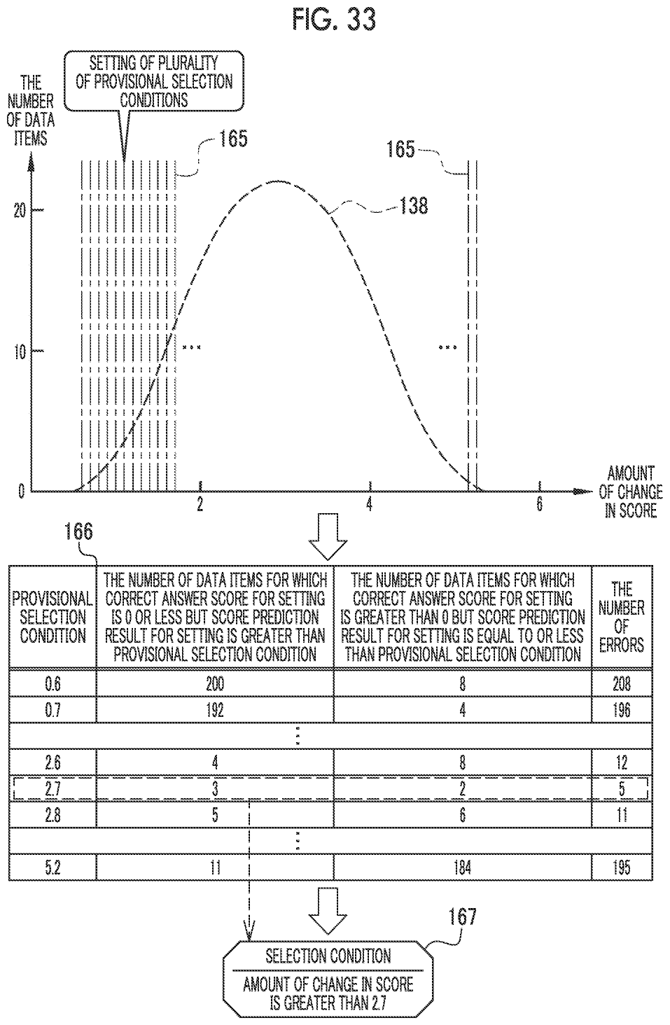

As shown in FIG. 32 as an example, in Method 3, a policy 160 is established by the user as to how many selection recommenders are to be secured as subjects for the clinical trial. Then, the amount of change indicated by a line 161 according to the policy 160 and drawn with respect to the score prediction result distribution for selection group setting 156 is set as a selection condition 162.

FIG. 32 exemplifies a case where the policy 160 of securing more than 80% of selection recommenders is established. In this case, the line 161 is a line that divides the score prediction result distribution for selection group setting 156 at a ratio of 2:8. The amount of change of 3.1 indicated by this line 161 is set as the selection condition 162. That is, the selection condition 162 has a content that the amount of change in the cognitive ability test score 25 is greater than 3.1.

As described above, in Method 3, the selection condition 162 is set based on the score prediction result distribution for selection group setting 156, which is the distribution of the number of data items of the score prediction result for setting 132S of the selection group, which is a group to be selected from the subject for the clinical trial, the group being extracted based on the correct answer score for setting 132SCA included in the clinical trial suitable data 131. Therefore, it is possible to secure a certain number of persons suitable as subjects for the clinical trial, that is, selection recommenders, as subjects for the clinical trial.

<Method 4>

As shown in FIG. 33 as an example, in Method 4, the amount of change in the cognitive ability test score 25 is changed in increments of 0.1 as indicated by a plurality of lines 165 in the score prediction result distribution for setting 138, whereby a plurality of provisional selection conditions are set. Then, as shown in Table 166, the number of errors in the score prediction result for setting 132S with respect to the correct answer score for setting 132SCA is counted for each of the plurality of provisional selection conditions. The number of errors is the sum of the number of data items for which the correct answer score for setting 132SCA is 0 or less but the score prediction result for setting 132S is greater than the provisional selection condition, and the number of data items for which the correct answer score for setting 132SCA is greater than 0 but the score prediction result for setting 132S is equal to or less than the provisional selection condition. The former case where the correct answer score for setting 132SCA is 0 or less but the score prediction result for setting 132S is greater than the provisional selection condition is a case where a person is actually an exclusion recommender but is selected as a selection recommender. On the other hand, the latter case where the correct answer score for setting 132SCA is greater than 0 but the score prediction result for setting 132S is equal to or less than the provisional selection condition is a case where a person is actually a selection recommender but is selected as an exclusion recommender.

Then, a provisional selection condition having a minimum number of counted errors is set as the selection condition 167. FIG. 33 exemplifies a case where the number of errors is the minimum 5 when the amount of change in the cognitive ability test score 25 of the provisional selection condition is 2.7. In this case, the selection condition 167 has a content that the amount of change in the cognitive ability test score 25 is greater than 2.7.

In this way, in Method 4, a plurality of provisional selection conditions are set in the score prediction result distribution for setting 138. Then, the number of errors in the score prediction result for setting 132S with respect to the correct answer score for setting 132SCA is counted for each of the plurality of provisional selection conditions, and the provisional selection condition having the minimum number of errors is set as the selection condition 167. Therefore, it is possible to further greatly reduce the probability of omitting a person suitable as a subject for the clinical trial from the selection, or conversely, selecting a person unsuitable as a subject for the clinical trial.

Note that a method of searching for the selection condition 167, the method disclosed in Document A or Document B described below may be used. The method of Document A or Document B is commonly used as a method of obtaining an optimum solution (here, the selection condition 167) from a plurality of candidates (here, a plurality of provisional selection conditions).
Document A: J Kittler, J Illingworth, J Foglein, Threshold selection based on a simple image statistic, Computer Vision, Graphics, and Image Processing, Vol 30, Issue 2, May 1985, pp. 125-147
Document B: Nobuyuki Otsu (1979). “A threshold selection method from gray-level histograms”. IEEE Trans. Sys. Man. Cyber. 9 (1): pp. 62-66.

<Method 5>

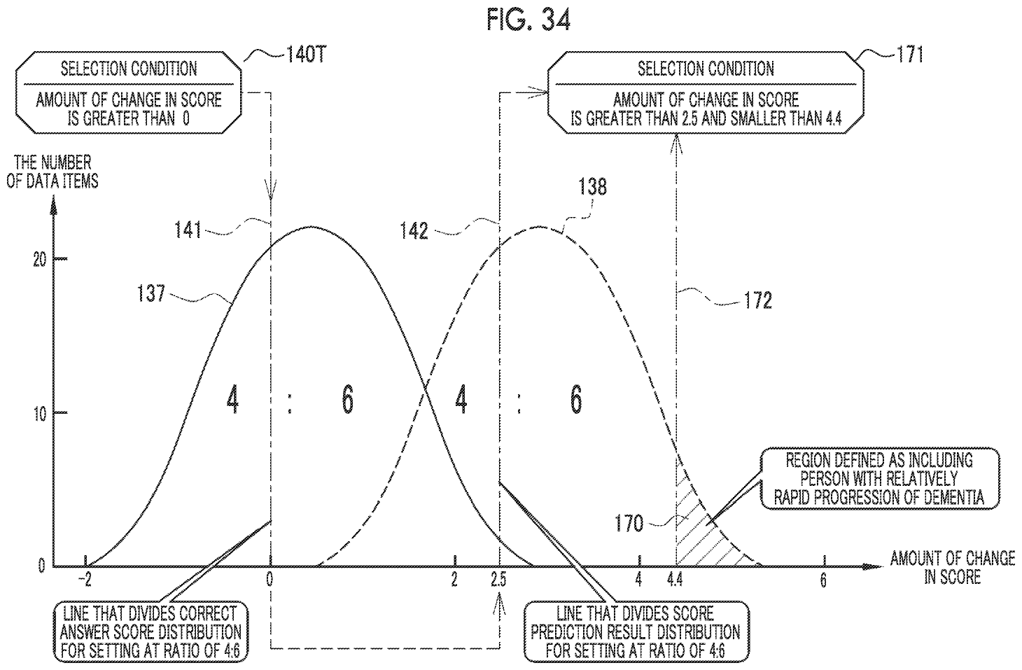

As shown in FIG. 34 as an example, Method 5 sets a selection condition 171 at a boundary of a region 170 defined as including a person with a rapid progression of dementia in the score prediction result distribution for setting 138. FIG. 34 exemplifies Method 1 shown in FIG. 26.

The region 170, more specifically, a line 172 of the boundary of the region 170 is defined by the user. The user may define the region 170 (line 172) based on the pharmacology of the anti-dementia drug or the result of the clinical trial, such as the animal experiment performed before the clinical trial using the dementia progression prediction model 41. The line 172 is, for example, a line drawn at a position separated from the average of the score prediction result distribution for setting 138 by +26 (a is a standard deviation) or +36. The amount of change of 4.4 indicated by the line 172 and the amount of change of 2.5 indicated by the line 142 are set as the selection condition 171. That is, the selection condition 171 has the content that the amount of change of the cognitive ability test score 25 is greater than 2.5 and smaller than 4.4.

In the case of a person with a rapid progression of dementia, the damage of the brain nerve is progressing, and the anti-dementia drug is likely to not work, and the efficacy of the anti-dementia drug cannot be correctly verified. Therefore, the person is not suitable as a subject for the clinical trial. Therefore, in Method 5, the selection condition 171 is set at the boundary of the region 170 defined as including a person with a rapid progression of dementia in the score prediction result distribution for setting 138. Thereby, it is possible to reduce the probability of selecting a person with a rapid progression of dementia as a subject for the clinical trial. Although Method 1 is exemplified in FIG. 34, Method 5 may be applied to Methods 2 to 4.

Although not shown, in Methods 2 to 5 as well, the determination unit 48 outputs the selection reference information 18 according to each of the selection conditions 152, 162, 167, and 171.

The process of inputting the target input data for setting 16S and the clinical trial period for setting 17S of the clinical trial suitable data 131 to the dementia progression prediction model 41 trained using the supervised training data 70 and outputting the score prediction result for setting 132S from the dementia progression prediction model 41 shown in FIG. 24 may be performed in the clinical trial subject selection support server 10, or may be performed by a device other than the clinical trial subject selection support server 10. In addition, the setting of the selection condition 140 by Method 1 shown in FIGS. 25 and 26, the setting of the selection condition 152 by Method 2 shown in FIGS. 29 and 30, and the setting of the selection condition 162 by Method 3 shown in FIGS. 31 and 32 may also be performed in the clinical trial subject selection support server 10, or may be performed by a device other than the clinical trial subject selection support server 10. Further, the setting of the selection condition 167 by Method 4 shown in FIG. 33 and the setting of the selection condition 171 by Method 5 shown in FIG. 34 may also be performed in the clinical trial subject selection support server 10, or may be performed by a device other than the clinical trial subject selection support server 10.

The clinical trial suitable data 131 may be prepared by the following method. That is, all the data 130 is divided into, for example, 80% of the supervised training data 70 and 20% of the test data. Then, data that satisfies the employment conditions is extracted as the clinical trial suitable data 131 from the test data.

The score prediction result is not limited to the amount of change described as an example. The probability of each of normal control, preclinical AD, mild cognitive impairment, and Alzheimer's dementia shown in FIG. 22 may be used. Further, the weighted sum of the amount of change and the probability of each of normal control, preclinical AD, mild cognitive impairment, and Alzheimer's dementia may be used.

Instead of distributing the selection reference information 18 from the clinical trial subject selection support server 10 to the user terminal 11, screen data and the like of the clinical trial subject selection support screen 85 shown in FIG. 16 or the like may be distributed from the clinical trial subject selection support server 10 to the user terminal 11.

The aspect of providing the selection reference information 18 for viewing by the drug discovery staff is not limited to the clinical trial subject selection support screen 85. A printed matter of the selection reference information 18 may be provided to the drug discovery staff, or an e-mail to which the selection reference information 18 is attached may be transmitted to the drug discovery staff's mobile terminal.

The learning of the dementia progression prediction model 41 shown in FIG. 8 may be performed in the clinical trial subject selection support server 10, or may be performed by a device other than the clinical trial subject selection support server 10. In addition, the learning of the dementia progression prediction model 41 may be continued even after the operation.

The clinical trial subject selection support server 10 may be installed in each pharmaceutical development facility or may be installed in a data center independent of the pharmaceutical development facility. In addition, the user terminal 11 may take some or all functions of each of the processing units 45 to 49 of the clinical trial subject selection support server 10.

The cognitive ability test score 25 may be a rivermead behavioural memory test (RBMT) score, an activities of daily living (ADL) score, or the like. Also, the cognitive ability test score 25 may be an ADAS-Cog score, an MMSE score, or the like.

The CSF test result 26 is not limited to the amount of p-tau 181 described as an example. The CSF test result 26 may be the amount of t-tau (total tau protein) or the amount of Δβ42 (amyloid β protein).

The MRI image 28 may be an image obtained by cutting out a portion of the brain, such as an image of a portion of a hippocampus. Also, a PET image or a SPECT image may be used as the test data 21 instead of or in addition to the MRI image 28.

As disclosed in WO2022/071158A, the score prediction result 50 may be output from the dementia progression prediction model 41 by, for example, extracting an image of an anatomical region of a brain, such as a hippocampus, from a medical image such as the MRI image 28, inputting the extracted image of the anatomical region to a feature amount derivation model such as a convolutional neural network to output the feature amount through a convolution operation or the like, and inputting the feature amount to the dementia progression prediction model 41 as the target input data 16. The feature amount well represents a shape of the anatomical region and a feature of a texture, such as a degree of atrophy of a hippocampus. Therefore, the prediction accuracy of the score prediction result 50 can be further improved. The image of the anatomical region to be extracted is not limited to the image of the hippocampus, and preferably includes a plurality of images of other anatomical regions, such as a parahippocampal gyms, a frontal lobe, an anterior temporal lobe (anterior part of a temporal lobe), an occipital lobe, a thalamus, a hypothalamus, and an amygdala. The image of the anatomical region to be extracted preferably includes at least an image of a hippocampus, and more preferably includes at least an image of a hippocampus and an image of an anterior temporal lobe. In this case, the feature amount derivation model is prepared for each of images of a plurality of anatomical regions. In this manner, the aspect of extracting an image of an anatomical region of a brain from a medical image, inputting the extracted image of the anatomical region to a feature amount derivation model to output the feature amount, and inputting the feature amount to the dementia progression prediction model 41 as the target input data 16 is particularly effective for predicting progression from MCI.

The prediction regarding dementia includes a prediction of a cognitive function, such as how much the cognitive function of the subject is reduced after, for example, two years, a prediction of a risk of developing dementia, such as a degree of the risk of developing dementia of the subject, and the like.

Although dementia has been exemplified as a disease, the present disclosure is not limited thereto. The disease may be, for example, cerebral infarction. The target input data 16 in this case includes a National Institutes of Health Stroke Scale (hereinafter abbreviated as NIHSS) score, a Japan Stroke Scale (hereinafter abbreviated as JSS) score, a CT image, an MRI image, and the like. In addition, the machine learning model is not limited to the machine learning model in which the plurality of types of target input data 16 related to the disease are input, such as the dementia progression prediction model 41. In this way, the medical support may be supported for selecting a clinical trial subject for diseases other than dementia. The disease may be cerebral infarction, which is exemplified, or neurodegenerative disease such as Parkinson's disease, and cranial nerve disease including cerebrovascular disease.

However, dementia has become a social problem with the advent of an aging society in recent years. For this reason, it can be said that the dementia progression prediction server 10 using the dementia progression prediction model 41 to which the target input data 16 related to dementia is input has a form that matches the current social problem.

In each of the above embodiments, for example, as hardware structures of processing units that execute various kinds of processing, such as the reception unit 45, the RW control unit 46, the prediction unit 47, the determination unit 48, and the distribution control unit 49, various processors shown below can be used. As described above, in addition to the CPU 32 which is a general-purpose processor that functions as various processing units by executing software (operation program 40), the various processors include a programmable logic device (PLD), which is a processor capable of changing a circuit configuration after manufacture, such as a field programmable gate array (FPGA), and a dedicated electrical circuit, which is a processor having a circuit configuration specifically designed to execute specific processing such as an application specific integrated circuit (ASIC).

One processing unit may be configured by one of the various processors, or may be configured by a combination of the same or different kinds of two or more processors (for example, a combination of a plurality of FPGAs and/or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system on chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the technology of the present disclosure, the above-described various embodiments and/or various modification examples may be combined with each other as appropriate. In addition, the present disclosure is not limited to each of the above-described embodiments, and various configurations can be used without departing from the gist of the present disclosure. Furthermore, the technology of the present disclosure extends to a storage medium that non-transitorily stores a program, in addition to the program.

The described contents and illustrated contents shown above are detailed descriptions of the parts related to the technology of the present disclosure, and are merely an example of the technology of the present disclosure. For example, the above description of the configuration, function, operation, and effect is an example of the configuration, function, operation, and effect of the parts according to the technology of the present disclosure. Therefore, needless to say, unnecessary parts may be deleted, new elements may be added, or replacements may be made to the described contents and illustrated contents shown above within a range that does not deviate from the gist of the technology of the present disclosure. Further, in order to avoid complications and facilitate understanding of the parts related to the technology of the present disclosure, descriptions of common general knowledge and the like that do not require special descriptions for enabling the implementation of the technology of the present disclosure are omitted, in the described contents and illustrated contents shown above.

In the present specification, the term “A and/or B” is synonymous with the term “at least one of A or B”. That is, the term “A and/or B” means only A, only B, or a combination of A and B. In addition, in the present specification, the same approach as “A and/or B” is applied to a case where three or more matters are represented by connecting the matters with “and/or”.

All documents, patent applications, and technical standards described in the present specification are incorporated in the present specification by reference to the same extent as in a case where each of the documents, patent applications, technical standards are specifically and individually indicated to be incorporated by reference.

What is claimed is:

1. A medical support device comprising:
a processor; and
a memory connected to or built into the processor,
wherein the processor is configured to:
acquire target input data which is input data related to a disease of a subject candidate for a clinical trial of a drug, and a clinical trial period;
input the target input data and the clinical trial period to a machine learning model trained using supervised training data including accumulated input data related to a disease at two or more points in time and a time interval of the input data, and cause the machine learning model to output a prediction result regarding the disease of the subject candidate in the clinical trial period; and
output selection reference information for determining whether or not to select the subject candidate as a subject for the clinical trial, according to the prediction result.

2. The medical support device according to claim 1,
wherein the time interval is an interval set according to the clinical trial period.

3. The medical support device according to claim 1,
wherein the input data includes at least one of test data indicating a result of a test related to a disease or diagnostic data indicating a result of a diagnosis related to the disease.

4. The medical support device according to claim 1,
wherein the machine learning model outputs, as the prediction result, a score quantitatively representing a degree of progression of a disease.

5. The medical support device according to claim 4,
wherein the machine learning model further outputs, as the prediction result, a class qualitatively representing the degree of progression of the disease.

6. The medical support device according to claim 1,
wherein, in addition to the supervised training data, clinical trial suitable data that satisfies an employment condition determined in advance according to the drug is provided,
a prediction result for setting from the machine learning model is output by inputting input data and a time interval of the clinical trial suitable data to the machine learning model, and
the processor is configured to output the selection reference information according to a selection condition set based on at least a prediction result distribution for setting, which is a distribution of the number of data items of the prediction result for setting.

7. The medical support device according to claim 6,
wherein the selection condition is set based on an exclusion group prediction result distribution, which is a distribution of the number of data items of the prediction result for setting of a group of persons to be excluded from the subject for the clinical trial, the group being extracted based on correct answer data included in the clinical trial suitable data.

8. The medical support device according to claim 6,
wherein the selection condition is set based on a selection group prediction result distribution, which is a distribution of the number of data items of the prediction result for setting of a group of persons to be selected as the subject for the clinical trial, the group being extracted based on correct answer data included in the clinical trial suitable data.

9. The medical support device according to claim 6,
wherein a plurality of provisional selection conditions are set in the prediction result distribution for setting, the number of errors in the prediction result for setting with respect to correct answer data is counted for each of the plurality of provisional selection conditions, and the provisional selection condition having a minimum number of errors is set as the selection condition.

10. The medical support device according to claim 6,
wherein the selection condition is set based on a correct answer data distribution, which is a distribution of the number of correct answer data items included in the clinical trial suitable data, in addition to the prediction result distribution for setting.

11. The medical support device according to claim 10,
wherein the selection condition is set by applying a provisional selection condition set in the correct answer data distribution to the prediction result distribution for setting.

12. The medical support device according to claim 6,
wherein the selection condition is set at a boundary of a region defined as including a person with a rapid progression of a disease in the prediction result distribution for setting.

13. The medical support device according to claim 1,
wherein the disease is dementia.

14. An operation method of a medical support device, the method comprising:
acquiring target input data which is input data related to a disease of a subject candidate for a clinical trial of a drug, and a clinical trial period;
inputting the target input data and the clinical trial period to a machine learning model trained using supervised training data including accumulated input data related to a disease at two or more points in time and a time interval of the input data, and causing the machine learning model to output a prediction result regarding the disease of the subject candidate in the clinical trial period; and
outputting selection reference information for determining whether or not to select the subject candidate as a subject for the clinical trial, according to the prediction result.

15. A non-transitory computer-readable storage medium storing an operation program of a medical support device causing a computer to execute a process comprising:
acquiring target input data which is input data related to a disease of a subject candidate for a clinical trial of a drug, and a clinical trial period;
inputting the target input data and the clinical trial period to a machine learning model trained using supervised training data including accumulated input data related to a disease at two or more points in time and a time interval of the input data, and causing the machine learning model to output a prediction result regarding the disease of the subject candidate in the clinical trial period; and
outputting selection reference information for determining whether or not to select the subject candidate as a subject for the clinical trial, according to the prediction result.
